(19) United States
(12) Patent Application Publication
(10) Pub. No.: US 2024/0361338 A1
(43) Pub. Date: Oct. 31, 2024

(54) DIAGNOSIS AND TREATMENT OF ECTOPIC ENDOMETRIOSIS

(71) Applicant: FERRING B.V., Hoofddorp (NL)

(72) Inventors: Torsten M. REINHEIMER, Copenhagen (DK); Glenn E. CROSTON, Davis, CA (US); Benedetta BUSSOLATI, Torino (IT)

(73) Assignee: FERRING B.V., Hoofddorp (NL)

(21) Appl. No.: 18/687,691

(22) PCT Filed: Aug. 30, 2022
(86) PCT No.: PCT/EP2022/074112

§ 371 Date: Feb. 28, 2024

Related U.S. Application Data
(60) Provisional application No. 63/239,260, filed on Aug. 31, 2021.

(30) Foreign Application Priority Data
Nov. 23, 2021 (EP) 21210034.1

(51) Int. Cl. G01N 33/68; A61K 31/473; A61P 15/00; C12N 5/00; C12N 5/0775; G01N 33/53

(52) U.S. Cl. CPC G01N 33/6896; A61K 31/473; A61P 15/00; C12N 5/0018; C12N 5/0662; G01N 33/53; C12N 2501/815; G01N 2333/70571; G01N 2333/70596; G01N 2333/71; G01N 2333/922; G01N 2800/364; G01N 2800/54; G01N 2800/56

(57) ABSTRACT

The present disclosure provides methods of detection and diagnosis of ectopic endometriosis. The disclosure further provides compositions, medicaments and methods for treating or preventing ectopic endometriosis.

### FIELD

The present disclosure provides compositions, medicaments and methods for treating or preventing ectopic endometriosis. The disclosure further provides methods of detection and diagnosis.

### BACKGROUND

Endometriosis is a chronic reproductive age-associated disease often characterized by the presence of ectopic endometrial tissue (that is, endometrial-like tissue outside the uterus: Galle et al., 1989; Giudice et al., 2004). Sex-steroid hormones are not only key players for the maintenance of normal uterine function and fertility, but they also regulate the growth of endometriotic lesions causing periodic bleeding and inflammation associated with pelvic pain and infertility (Gibson et al., 2020). Importantly, stem cell activity in the basalis of the endometrium plays a critical role in endometrial function, supporting cyclic regeneration after menstruation (Schwab et al., 2005; Masuda et al., 2010; Gargett et al., 2016). In particular, local endometrial mesenchymal stromal cells (E-MSCs) have been isolated and characterized in several works (Gargett et al., 2016 bis), stimulating interest in their potential in endometrium regeneration. E-MSCs recapitulate the majority of bone marrow mesenchymal stem cell properties (Dominici et al., 2006), including clonogenicity, multipotency and a specific surface phenotype that distinguish them from leukocytes, hematopoietic and endothelial cells (Dimitrov et al., 2008; Gargett et al., 2009). Moreover, E-MSCs show strong self-renewal in vitro (Gargett et al., 2009) and a capacity to regenerate endometrial stromal vascular tissue in vivo (Masuda et al., 2010; Cervellò et al., 2011). In addition, E-MSCs represent a heterogenic population of mesenchymal stem cells and stromal fibroblast, sharing a number of markers and functions. The specific expression of CD146, platelet-derived growth factor-receptor beta (PDGFRβ) and sushi domain containing-2 (SUSD2) revealed E-MSCs pericyte identity and perivascular localization, respectively (Schwab et al., 2007; Masuda et al., 2012).

According to the guidelines provided by the European Society of Human Reproduction and Embryology, medical treatment for endometriosis-associated pain and infertility is based on the administration of anti-inflammatory drugs coupled with the agents acting on the hormonal alteration of the menstrual cycle to produce chronic anovulation and an overall hypoestrogenic environment (Dunselman et al., 2014). However, as E-MSCs lack oestrogen receptors, endometriosis eradication cannot be achieved by hormone-based pharmacological approaches. Anti-angiogenic drugs are currently of increasing interest in consideration of the role of vasculogenesis and angiogenesis in progression and maintenance of endometriotic lesions (Pittatore et al., 2014).

Indeed, the pro-angiogenic environment has a critical role in the implantation, maintenance and growth of endometriotic implants, as supported by the increase in vascular endothelial growth factor (VEGF) levels in the peritoneal fluid of women with endometriosis and in endometrial tissue (Donnez et al., 1998; McLaren et al., 2000; Bourlev et al., 2006; Liu et al., 2020). E-MSCs isolated from endometriotic lesions are able to release large amounts of angiogenic factors like VEGF, as previously observed (Moggio et al, 2012, Liu et al., 2020). In this context, dopamine and its receptor agonists may represent an alternative to current antiangiogenic agents due to the inhibition of VEGF release and VEGF receptor 2 (VEGFR-2) activation (Sinha et al., 2009). Among the non-ergot D₂ agonist, quinagolide has been successfully tested in an experimentally induced endometriosis rat model (Akyol et al., 2016), and quinagolide tablets are marketed for treatment of hyperprolactinemia for a long time with substantial clinical experience and safety data (Barlier et al, 2006). Moreover, quinagolide is currently undergoing two different phase 2 trials investigating the effect of drug-releasing vaginal rings in women with endometriosis (NCT03749109, NCT03692403). However, its pivotal efficacy on E-MSCs is still unknown. Recently it was shown that the dopamine receptor type 2 (D₂) agonist cabergoline treatment reduced the angiogenic potential of ectopic E-MSCs in an endothelial co-culture setting (Canosa et al., 2017). In the present study, the effects of quinagolide treatment on eutopic and ectopic E-MSC lines isolated from ovarian and peritoneal lesions, and the related molecular mechanisms involved were evaluated.

The use of quinagolide for the treatment of endometriosis is suggested in patents WO2016/071466, US2012/0157489, US2010/0113499 and US2008/0293693. In particular, US2012/0157489 proposes the treatment of endometriosis by the delivery of quinagolide by vaginal administration, for example by vaginal pessary or tablet, or by vaginal ring.

### SUMMARY

The term “endometriosis” is a general term embracing a highly variable spectrum of conditions. For example, the term “endometriosis” is often used to describe disorders which may otherwise be described as “adenomyosis”. Adenomyosis differs from endometriosis in that it is defined by the presence of endometrial tissue within the myometrium. The term “endometriosis” may embrace occurrences of “ectopic endometriosis” in which endometrial tissue makes its way outside of the uterus and grows on or within other organs or structures including, for example, the ovaries, fallopian tubes, the cavities of the pelvis, the supporting ligaments of the uterus or the peritoneum.

This disclosure relates to those conditions characterised by the growth of endometrial tissue outside of the uterus and which may be collectively referred to as occurrences of “ectopic endometriosis”.

The disclosure is based partly on the finding that ectopic E-MSCs express more of the dopamine receptor D₂ (D₂) than eutopic E-MSCs. Without being bound by theory, this differential expression makes ectopic E-MSCs more sensitive to the action of, for example, a D₂ agonist.

Accordingly, the disclosure provides a D₂ agonist for use in treating or preventing ectopic endometriosis.

The disclosure also provides the use of a D₂ agonist or a D₂ agonist for use, in inhibiting or preventing the invasive properties of ectopic E-MSCs.

Also disclosed is the use of a D₂ agonist or a D₂ agonist for use, in inhibiting, limiting or preventing the endothelial differentiation of ectopic E-MSCs. The inhibition, limiting or prevention of endothelial differentiation of ectopic E-MSCs may be identified or observed in an endothelial co-culture model of angiogenesis.

A D₂ agonist may also be used to contain, control, restrict or inhibit the growth, development or spread of ectopic endometriosis. A D₂ agonist may be used to prevent the spread of endometriosis outside the uterine cavity—this may help prevent occurrences of ectopic endometriosis.

The term D₂ agonist may include, for example selective D₂ agonist and specific drugs such as ergoline D₂ agonists, non-ergoline D₂ agonists, pramipexole, ropinirole, rotigotine, bromocriptine, octreotide, carbergoline and quinagolide.

It should be noted that at least some of the teachings and embodiments of this disclosure are described in relation to the D₂ agonist, quinagolide; however, any (selective) D₂ agonist may be used—including any of those mentioned above. Moreover, it should be noted that the terms “comprise”, “comprising” and/or “comprises” is/are used to denote that aspects and embodiments of this invention “comprise” a particular feature or features. It should be understood that this/these terms may also encompass aspects and/or embodiments which “consist essentially of” or “consist of” the relevant feature or features.

Quinagolide (C₂₀H₃₃N₃O₃S) is a selective, D₂ agonist with a molecular mass of about 395 g/mol. Quinagolide is used for the treatment of elevated levels of prolactin.

It is commercially available under the trade name NORPROLAC® and is manufactured and used in the form of the hydrochloride salt. Quinagolide has the formula:

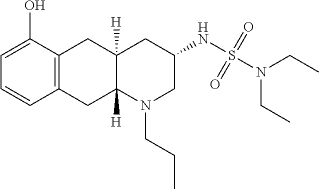 [SMILES: CCCN1C[C@@H](NS(=O)(=O)N(CC)CC)C[C@@H]2Cc3c(O)cccc3C[C@H]21]

and is N,N-diethyl-N′-[(3S,4aS,10aR)-6-hydroxy-1-propyl-1,2,3,4,4a,5,10,10a-octahydrobenzo[g]quinolin-3-yl]sulfamide.

The use of quinagolide to treat hyperprolactinemia is disclosed in Eur. J. Endocrinol Feb. 1, 2006 154 page 187-195.

As used herein, the term “quinagolide” includes all commercially available forms as well as functional derivatives and variants thereof. The term “quinagolide” also embraces all pharmaceutically acceptable (and active) salts and esters, including, for example, quinagolide hydrochloride. Quinagolide hydrochloride is a white crystalline powder of high melting point (231-237° C. under decomposition), that is sparingly soluble in water.

The term “quinagolide” also embraces any identified active enantiomers (for example the (−) enantiomer (see formula 1 below). As shown in Formula 1 and 2 below, quinagolide hydrochloride (C₂₀H₃₃N₃O₃S, HCl) is a racemate containing the two enantiomers with absolute configuration (3S,4aS,10aR) and (3R,4aR,10aS) respectively in a 1:1 ratio.

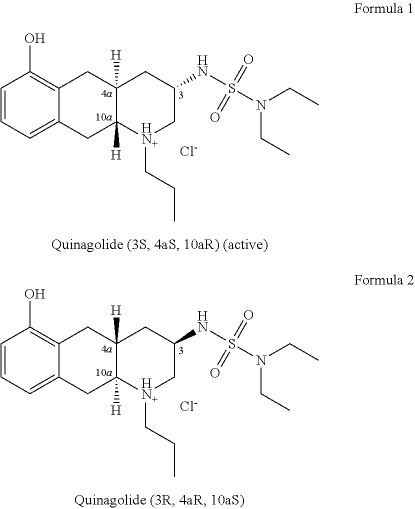

The two main metabolites of quinagolide, N-desethyl (also referred to as M1 or SDZ 214-368) and N,N-didesethyl (also named M2 or SDZ 214-992), may have similar D₂ binding affinity and potency as quinagolide; as such, the term “quinagolide” as used herein, may extend to quinagolide metabolites-including, for example the M1 and M2 metabolites. The term may extend to any quinagolide analogue or derivative that is metabolised in vivo to either or both of the M1 or M2 metabolites.

Given that the quinagolide (M1/M2) metabolites are active (and therapeutically useful), the term ‘quinagolide’ might extend to quinagolide (M1/M2) metabolites (or indeed any other of the active quinagolide salts, derivatives or enantiomers described herein).

For the avoidance of doubt, the general term “quinagolide” as used herein, relates to all of the forms, enantiomers, salts, metabolites and derivatives described herein.

Endometrial mesenchymal stromal cells (E-MSCs) extensively contribute to the establishment and progression of endometrial ectopic lesions, through formation of the stromal vascular tissue and support of its growth and vascularization. The data disclosed herein provides an insight into the effect of quinagolide on E-MSCs isolated from eutopic endometrial tissue and ectopic endometriotic lesions. Without wishing to be bound to any particular finding or theory, it is noted that the data disclosed herein shows that E-MSCs express D₂, with higher expression in ectopic E-MSCs.

Quinagolide was shown to inhibit the invasive properties of E-MSCs and to limit their endothelial differentiation in an endothelial co-culture model of angiogenesis. The abrogation of the observed effects by spiperone, a dopamine antagonist, confirmed specific D₂ activation, and no involvement of VEGFR-2 inhibition was observed. Moreover, D₂ activation led to downregulation of AKT and its phosphorylation. Of interest, several effects were more prominent on ectopic E-MSCs compared to eutopic lines.

Accordingly, the present disclosure provides quinagolide for use in the treatment or prevention of ectopic endometriosis.

The disclosure further provides a method of treating or preventing ectopic endometriosis, said method comprising administering a subject in need thereof, a therapeutically effective amount of quinagolide.

The disclosure also provides use of quinagolide in the manufacture of a medicament for the treatment and/or prevention of ectopic endometriosis.

Without wishing to be bound by theory, the data suggests that treatment with quinagolide reduces the invasive and/or angiogenic properties of E-MSCs, including ectopic E-MSCs. The data further suggests that quinagolide is able to limit E-MSC/ectopic E-MSC endothelial differentiation (as may be observed in an endothelial co-culture model of angiogenesis). Again, without wishing to be bound by theory, at least the observed anti-invasive effect of quinagolide may be D₂ dependent and therefore, this disclosure provides:
Use of quinagolide or quinagolide for use in inhibiting or reducing E-MSC/ectopic E-MSC invasion, angiogenesis and/or endothelial differentiation; and
Use of quinagolide or quinagolide for use in inhibiting or reducing E-MSC/ectopic E-MSC invasion, angiogenesis and/or endothelial differentiation in ectopic endometriosis; and
A method of inhibiting the invasive, angiogenic and/or endothelial differentiation properties of E-MSCs/ectopic E-MSCs, said method comprising contacting an E-MSC/ectopic E-MSC or administering a subject in need thereof, an invasion, angiogenesis and/or endothelial differentiation inhibiting amount of quinagolide; and
A method of treating or preventing ectopic endometriosis by inhibiting the invasive, angiogenic and/or endothelial differentiation properties of E-MSCs/ectopic E-MSCs, said method comprising administering a subject in need thereof a therapeutically effective amount of quinagolide.

As stated, ectopic endometriosis is an invasive disease effecting tissues, organs and structures outside of the uterus. Given the effect of quinagolide on the invasive, angiogenic and differentiation properties of E-MSCs, including ectopic E-MSCs, the disclosure provides quinagolide for use in containing, limiting and/or inhibiting the spread of ectopic endometriosis. The disclosure further provides a method of containing, limiting and/or inhibiting the spread of ectopic endometriosis by inhibiting the invasive and/or angiogenic properties of E-MSCs/ectopic E-MSCs (these cells contributing to the pathology of the disease), said method comprising administering a subject in need thereof a therapeutically effective (or invasion/angiogenesis limiting/inhibiting) amount of quinagolide. In this case, the subject may be experiencing an active and/or developing a case of endometriosis. The treatment of such subjects with quinagolide may help limit and contain the spread of the ectopic endometriosis by inhibiting the invasive, angiogenic and/or differentiation properties of the E-MSC/ectopic E-MSCs which cause or contribute to the pathology of the disease. Quinagolide may be formulated for different modes of administration and one of skill will appreciate that the precise formulation may vary depending on the route of administration.

Any mode of administration that provides the desired therapeutic effect may be suitable for use according to the present disclosure. Such modes of administration include oral, intranasal, rectal, topical, transdermal, sublingual, intramuscular, parenteral, intravenous, intracavity, vaginal, and adhesive matrix to be used during surgery. By way of particular example, the quinagolide may be formulated for intravaginal administration.

Quinagolide may be used in the form of a pharmaceutical composition. In such compositions, the quinagolide may be formulated together with one or more pharmaceutically acceptable excipients, diluents and/or carriers. Pharmaceutical compositions for use in the methods described herein may be prepared conventionally, comprising substances that are customarily used in pharmaceuticals and as described in, for example, Remington's The Sciences and Practice of Pharmacy, 22nd Edition (Pharmaceutical Press 2012), Pharmaceutics—The Science of Dosage Form Design, Churchill Livingston (1988) and/or Handbook of Pharmaceutical Excipients, Ninth edition (Pharmaceutical Press, 2020) —the entire content of all of these documents being incorporated by reference.

By way of example only, suitable excipients may be selected from natural polymers, cellulose (such as microcrystalline cellulose), and derivatives thereof (such as ethyl cellulose, (hydroxypropyl)methyl cellulose (HPMC) and hydroxypropyl cellulose (HPC)). Other excipients that may be used include polysaccharides (such as pregelatinised starch and pullulan), Zein), polyvinylpyrrolidone (PVP), silica, metal stearates (e.g. magnesium stearate), and lactose. For example, quinagolide may be formulated with microcrystalline cellulose (such as Avicil®) and ethyl cellulose.

Suitable compositions containing quinagolide may comprise commercially available compositions, such as NORPROLAC® and/or those described in WO2010150098, the contents of which are incorporated by reference.

As noted above, quinagolide may be formulated for intravaginal administration. In some examples, quinagolide may be formulated as a tablet or capsule for intravaginal administration. In other examples, quinagolide may be comprised within and/or loaded into a polymeric drug-device unit. In other words, the pharmaceutical composition may be the polymeric drug-device unit.

As used herein, a polymeric drug-device unit is to be construed as a device which contains one or more drugs or active ingredients/agents comprised within, loaded into and/or dispersed in a polymer composition to form the polymeric drug-device unit. The term “drug-device unit” is intended to mean a combination product of drug and device/carrier where the device or carrier may act (actively or passively) by virtue of its design, physical characteristics and/or formulation properties, to allow release the drug in a controlled fashion. A drug-device unit of this disclosure may be an integrated unit which may comprise a drug (active agent) loaded polymeric system or a drug (active agent) loaded polymeric device which, in use is capable of dispensing and/or eluting an active agent. The drug-device units of this disclosure may be for the controlled and/or sustained delivery of an active agent.

As such, the polymeric drug-device unit for use in the methods of the present disclosure may comprise a polymer composition and quinagolide. Such polymeric drug-device units find particular application in the controlled and/or sustained delivery of quinagolide to a subject in need thereof (e.g. in the vaginal cavity) and/or may minimise an initial burst release of quinagolide. As used herein, the term “burst release” referring to a rapid and/or uncontrolled release of a pharmaceutically active agent from a polymeric drug-device unit over a relatively short period of time.

Suitable polymer compositions for use in the described polymeric drug-device units include those that are capable of one or more of:
(i) being loaded with quinagolide;
(ii) storing quinagolide over a period of time (e.g. an extended period of time);
(iii) allowing for a controlled release and/or sustained delivery of quinagolide; and/or
(iv) limiting, controlling and/or minimising an initial burst release of quinagolide.

Furthermore, suitable polymer compositions may include those that exhibit mechanical properties that suit, facilitate or permit use, location and/or retention in a vaginal cavity.

Polymers compositions for use in such polymeric drug-device units may be polyurethanes, e.g. polyurethane block copolymers. Suitable polymeric drug-device units comprising quinagolide are described in WO 2016/071466, the entire contents of which are incorporated herein by reference.

Other polymer compositions that may be suitable for use in forming the polymeric drug-device units include those described in WO2009/094573, WO2010/019226, WO2004/096151, U.S. Pat. No. 4,235,988 and WO2005/004837, WO2013/013172, WO2008/007046, and WO2012/066000. The entire contents of these documents are incorporated herein by reference.

By way of example only, a polyurethane block copolymer for use in the polymeric drug-device units disclosed herein may be obtainable or may be obtained by reacting together:
(a) a poly(alkylene oxide);
(b) a difunctional compound;
(c) a difunctional isocyanate; and
(d) optionally a block copolymer comprising poly(alkylene oxide) blocks.

Component (a) may comprise one or more poly(alkylene oxide)s. Poly(alkylene oxide)s contain the repeating ether linkage —R—O—R— and can have two or more hydroxyl groups as terminal functional groups. These polymers are also referred to as polyalkylene glycols or polyglycols. The poly(alkylene oxide) may be a polyethylene glycol (PEG), a polypropylene glycol (PPG), a poly(tetramethylene oxide) (PTMO) or poly(hexamethylene oxide) (PHMO). The poly(alkylene oxide) may be polypropylene glycol.

Polyethylene glycols contain the repeat unit (CH₂CH₂O) and can have the structure HO(CH₂CH₂O)ₙH wherein n is an integer of varying size depending on the molecular weight of the polyethylene glycol. Polyethylene glycols used in the present disclosure are generally linear polyethylene glycols and/or generally have a molecular weight of 200 to 35,000 g/mol, particularly 1,000 to 10,000 g/mol and especially 1,500 to 5,000 g/mol. For example, the polyethylene glycol may have a molecular weight of approximately 2,000 g/mol.

Polypropylene glycols contain the repeat unit (CH₂CH(CH₃)O) and can have the structure HO(CH₂CH(CH₃)O)ₙH, wherein n is an integer of varying size depending on the molecular weight of the polypropylene glycol. Polypropylene glycols used in the present disclosure are generally linear polypropylene glycols and/or generally have a molecular weight of 200 to 35,000 g/mol, particularly 1,000 to 10,000 g/mol and especially 1,500 to 5,000 g/mol. For example, the polypropylene glycol may have a molecular weight of approximately 2,000 g/mol.

Polyurethane block copolymers used in the present disclosure may be obtainable by also reacting a block copolymer comprising a poly(alkylene oxide) block together with the components (a), (b) and (c). The block copolymer comprising a poly(alkylene oxide) block may be a poly(alkylene oxide) block copolymer. The block copolymer may comprise blocks of polyethylene glycol, polypropylene glycol, a poly(tetramethylene oxide) (PTMO), poly(hexamethylene oxide) (PHMO), and/or polysiloxanes, such as polydimethylsiloxane (PDMS). The block copolymer may comprise blocks of polyethylene glycol and polypropylene glycol. PEG-PPG-PEG and PPG-PEG-PPG copolymers used in the present disclosure are generally linear having molecular weights in the range 200 to 14,000 g/mol. For example, the PEG-PPG-PEG and PPG-PEG-PPG block copolymers used in the present disclosure may have a molecular weight of approximately 2,000 g/mol.

As will be appreciated, the PEG content in the block copolymer may be varied. For example, a PEG-PPG-PEG copolymer may be used that comprises approximately 10% by weight of PEG. In other examples, a PPG-PEG-PPG copolymer may be used that comprises approximately 50% by weight of PEG. These exemplary block copolymers are typically commercially available. However, it will be appreciated that block copolymers having alternative compositional ranges may be used to provide pharmaceutical delivery devices according to the disclosure.

Unless the context indicates otherwise, references to a polymer “molecular weight” may refer to a “number average molecular weight”. Any references to an “equivalent weight” may refer to the number average molecular weight divided by the functionality of the compound. As used herein, the number average molecular weight of a polymer is the mean molecular weight of the polymer. The number average molecular weight may be calculated by summing the molecular weights of n polymer molecules and dividing by n. A variety of techniques may be used to determine the number average molecular weight of a polymer. Representative examples of such techniques include, but are not limited to, gel permeation chromatography, viscometry, and proton-NMR.

Component (b) may comprise one or more difunctional compound(s). The difunctional compound is reactive with the difunctional isocyanate. Suitable difunctional compounds include, for example, diols, diamines and amino alcohols.

Generally, a short chain diol is used as the difunctional compound. For example, diols in the range C₃ to C₂₀, particularly C₄ to C₁₀, especially C₄ to C₆ may be used. The diol may be a saturated or unsaturated diol. Branched or straight chain diols may be used. Representative examples of suitable diols include (but are not limited to) 1,4-butanediol, 1,5-pentanediol, 1,6-hexanediol, 1,10-decanediol, 1,12-dodecanediol and 1,16-hexadecanediol.

Component (c) may comprise one or more difunctional isocyanate(s). The difunctional isocyanate may be an aromatic diisocyanate, such as diphenylmethane-4,4′-diisocyanate. The difunctional isocyanate may be an aliphatic diisocyanate, such as dicyclohexylmethane-4,4′-diisocyanate (DMDI), hexamethylene diisocyanate (HMDI) etc. In one embodiment, the difunctional isocyanate is DMDI.

In general, the combined molar ratio of starting components (a), (b) and (d) should equal the molar ratio of starting component (c). Adhering to this general principle may ensure a balanced stoichiometry and facilitate complete (or substantially complete) reaction of all the starting polymer components. During the reaction of the starting components, one or more reaction parameters may be monitored to assess the stoichiometry and/or progress of the reaction/consumption of the starting components. The molar ratio of the components (a) to (b) to (c) is generally in the range 0.05-0.75 to 1 to 1.00-2.00. In those cases where component (d) is also present, the ratio of components (a) to (b) to (c) to (d) is generally in the range 0.05-0.75 to 1 to 1.00-2.00 to 0.01-0.50. In some cases, the molar ratio may be in the range 0.05-0.25 to 1 to 1.05-1.5 to 0.025-0.30. In other cases, the molar ratio of components may be in the range 0.05-0.20 to 1 to 1.1-1.4 to 0.03-0.25. For example, the molar ratio of components may be approximately 0.16 to 1 to 1.21 to 0.06.

As will be appreciated by a skilled person, the above molar ratios of components are based on components (a) and (d) having idealised molecular weights. By way of example, where component (a) is PPG and/or component (b) is a PPG-PEG-PPG block copolymer, the above molar ratios apply to each of those components having an idealised molecular weight of 2,000. Accordingly, to ensure the weight percentages of components (a), (b), (c) and optionally (d) remain consistent in the polyurethane block copolymer when using different raw materials, the skilled person may adjust the molar ratio as appropriate (e.g. after ascertaining the exact number average molecular weight of components (a) and (d)).

By way of representative example only, the polymer composition may be as set out below:

| Polymer | | Stoichiometry |
|---|---|---|
| Batch | Starting polymer composition (wt %) | (a):(b):(c):(d) |
| Example 1 | PPG2000 37.0%; pentanediol 12.3%; | 0.156:1:1.211:0.056 |
| | DMDI 37.7%; PPG-PEG-PPG2000 | |
| | 13.0% | |

The polymeric drug-device units disclosed herein may take any form that is suitable for administration into vaginal cavity e.g. a vaginal pessary or vaginal ring. By way of example only, the polymeric drug-device unit may be generally toroidal in shape and/or may be formed of joined tubular lengths of polymer. Such rings may be sized and dimensioned such that they may be inserted, located and/or retained in the vaginal cavity.

The polymeric drug-device units may comprise quinagolide (or a pharmaceutically acceptable salt thereof: for example, quinagolide hydrochloride) at an amount or dose of about 25 to about 15,000 micrograms (μg), or about 200 to 5,000 μg. For example, the polymeric drug-device unit may comprise quinagolide at a dose of about 400-3,000 μg. Typically, about 200 μg, about 400 μg, about 800 μg, about 1200 μg, about 1500 μg about 2400 μg and about 3000 μg quinagolide is contained (or dispersed) within a polymeric drug-device unit of this disclosure.

In use, the polymeric drug-device units may demonstrate or achieve a continuous release of quinagolide to the vaginal tissues. One of skill will appreciate that the magnitude or amount of quinagolide continuously released from the polymeric drug-device units will vary depending on the amount loaded into and/or dispersed within the polymeric drug-device unit. Typically, the release may be steady and constant over a particular/predetermined time. The polymeric drug-device unit may continuously release anywhere between about 1 and about 100 μg, 150 μg or 350 μg quinagolide/day; for example, 1 and about 50 μg quinagolide/day. Depending on the formulation (and perhaps other factors) the polymeric drug-device unit may continuously release about 5, about 10, about 15, about 20 or about 30 μg quinagolide/day. The polymeric drug-device unit may continuously release at least about 5, at least about 10, at least about 15, at least about 20 or at least about 30 μg quinagolide/day. As another example, the drug-device unit may continuously release about 45, about 40, about 35, about 30 or about 25 μg quinagolide/day.

The release of quinagolide from a drug-device unit as described herein may be assessed, monitored or determined using methods or protocols which determine the release of quinagolide in a dissolution medium (a buffer, such as water) at some predetermined temperature or temperatures—for example at about 37° C. (±0.5° C.). A suitable protocol may use a volume of water which is appropriate to ensure sink conditions for release of the analyte (in this case the “quinagolide”). A sample (for example a sample or test drug-device unit as described herein) may be contained in a closed vessel, for example a Duran® flask or the like, for a predetermined period of time (for example about 35 days—however the precise time may vary depending on the conditions and protocol). The closed vessels may be agitated and/or shaken/stirred for set or extended periods of time throughout the protocol.

Alternatively or additionally, the polymeric drug-device units may provide a therapeutically effective plasma concentration of quinagolide in a patient without (or substantially without, or minimising) adverse and/or toxic effects. For example, the drug-device units may be formulated such that the plasma concentration of quinagolide is at or below some predetermined safe (non-toxic level). For example (and without wishing to be bound by theory or examples), the polymeric drug-device units may provide a concentration of quinagolide of less than or equal to about 50 pg/ml in the plasma. The polymeric drug-device units may provide a substantially constant level of quinagolide in the blood plasma of between about 1 and 100 pg/ml or between about 1 and 50 pg/ml, e.g. between about 1 and 20 pg/ml over an extended period of time (e.g. over 21 days, over 28 days or over 35 days). The substantially constant plasma concentration of quinagolide may be achieved within 1 to 48 hours (for example by about 36 to about 46 hours (or higher (in the original patient values)) after administration.

The polymeric drug-device units may modulate an initial burst release or a steady-state release of quinagolide within 12-36 hours (e.g. within about 24 hours) after initial administration of the polymeric drug-device unit. Further, the polymeric drug-device units may provide a substantially constant level of quinagolide in the blood plasma of a subject over an extended period of time (e.g. over 21 days, over 28 days or over 35 days).

The polymeric drug-device units may restrict or contain an initial burst release of an active agent relative to the steady state release of that agent. A quotient calculated by dividing the percentage release over an initial 24 hour period by the percentage release over a later period (e.g. a period equating to 7-14 days after administration) may provide an indication of the relative magnitude of the burst release. For example, a lower release quotient may indicate a reduced burst release relative to the steady state release. Certain of the polymers described herein may provide a quotient between 0.05 and 10. In some examples, the polymer compositions may provide quotients between about 0.1 and 0.5, or between 0.2 and 0.4.

The present disclosure provides methods for detecting ectopic endometrial mesenchymal stromal cells (E-MSCs). Relative to eutopic E-MSCs, ectopic E-MSCs express different cell surface molecules—in other words, they express or possess different cell-surface profiles. In particular it has been noted that as compared to eutopic E-MSCs, ectopic E-MSCs express relatively higher amounts of D₂. It has additionally been noted that as compared to eutopic E-MSCs, ectopic E-MSCs express relatively higher amounts of endoglin (CD105, a type I membrane glycoprotein that is part of the TGFβ receptor complex) and relatively lower amounts of platelet derived growth factor receptor beta PDGFRβ (CD140b).

Accordingly, this disclosure provides methods by which one may be able to identify or detect ectopic E-MSCs. Such methods may be used to detect the presence or absence of ectopic E-MSCs in samples, including clinical samples. Moreover, methods of detecting ectopic E-MSCs may find application in the diagnosis and treatment/prevention of ectopic endometriosis.

The various methods described herein may be based on the detection and/or analysis of D₂ expression within a sample. The various methods described herein may further involve the detection and/or analysis of endoglin and/or PDGFRβ expression within a sample.

The term “sample” may embrace any biological samples, such as biological fluids and/or tissues. A sample may comprise a biopsy, blood (or a fraction thereof (serum/plasma)), secretions, scrapings, tissue or organ washes or cells.

Subjects from whom samples may be provided or obtained include, for example, healthy subjects showing no detectable signs of ectopic endometriosis.

Samples may also be provided by, or obtained from, subjects suffering from or susceptible/predisposed to ectopic endometriosis. The subjects may be asymptomatic and/or symptomatic. The sample may be obtained from or provided by subjects between recognised and/or diagnosed instances of ectopic endometriosis.

A sample may be a stored or preserved sample.

The sample may comprise a cell, for example an E-MSC.

A sample of any type described herein and for use with a method of this disclosure may be referred to as a “test sample”.

A method for the detection of an ectopic E-MSC may comprise determining a level of D₂ expression within a test sample. For example, the method may be applied to an E-MSC cell (or a sample comprising (or thought to comprise) the same), wherein the level of D₂ expression will determine whether or not (1) that cell is an ectopic E-MSC and/or (2) the sample comprises an ectopic E-MSC.

In one teaching, the disclosure provides a method of detecting an ectopic E-MSC in a sample, said method comprising determining a level of D₂ expression in the sample.

The results of these methods may be compared to a control or reference amount of D₂. The control or reference amount may provide a known or predetermined amount of D₂. The control or reference amount may comprise or be derived from a eutopic E-MSC or a sample comprising eutopic E-MSCs, wherein relative to the amount of D₂ expressed by an ectopic E-MSC or a sample comprising ectopic E-MSCs, eutopic E-MSCs have now been shown to express a lower amount of D₂.

In such cases, the amount of D₂ detected in the sample (the ‘test’ sample) can be compared with the reference or control amount of D₂. An ectopic E-MSC or the presence of ectopic E-MSCs within a test sample will be determined by a level of D₂ which is higher than that of the control/reference amount.

Additionally or alternatively, the method for the detection of an ectopic E-MSC may comprise determining a level of CD105 expression within a test sample. For example, the method may be applied to an E-MSC cell (or a sample comprising the same), wherein the level of CD105 expression will determine whether or not (1) that cell is an ectopic E-MSC and/or (2) the sample comprises a E-MSC.

In one teaching, the disclosure provides a method of detecting an ectopic E-MSC in a sample, said method comprising determining a level of CD105 expression in the sample.

The results of these methods may be compared to a control or reference amount of CD105. The control or reference amount may provide a known or predetermined amount of CD105. The control or reference amount may comprise or be derived from a eutopic E-MSC or a sample comprising eutopic E-MSCs, wherein relative to the amount of CD105 expressed by an ectopic E-MSC or a sample comprising ectopic E-MSCs, eutopic E-MSCs have now been shown to express a lower amount of CD105.

In such cases, the amount of CD105 detected in the sample (the ‘test’ sample) can be compared with the reference or control amount of CD105. An ectopic E-MSC or the presence of ectopic E-MSCs within a test sample will be determined by a level of CD105 which is higher than that of the control/reference amount.

Additionally or alternatively, the method of detecting an E-MSC or a test sample comprising the same, may further comprise a step in which a cell (for example a E-MSC) or the test sample is alternatively or additionally probed for the presence of CD140b.

Again, the level or amount of CD140b expressed by the E-MSC or detected in the test sample may be compared to a control/reference amount of CD140b. The control or reference amount/assay may provide a known or predetermined amount of CD140b. The control or reference amount may comprise or be derived from a eutopic E-MSC or a sample which comprises eutopic E-MSCs, wherein relative to the amount of CD140b expressed by an ectopic E-MSC or a sample comprising ectopic E-MSCs, eutopic E-MSCs have now been shown to express a higher amount of CD140b.

In such cases, the amount of CD140b detected in the sample (the ‘test’ sample) can be compared with the reference/control amount/level of CD140b. An ectopic E-MSC or a sample comprising ectopic E-MSCs will be determined by a level of CD140b which is lower than the control/reference amount.

A step of detecting or identifying an ectopic E-MSC or a sample comprising the same, may further comprise a step or steps in which a test cell or test sample is further or additionally probed for the presence or expression of one or more mesenchymal, haematopoietic, endometriotic, epithelial and/or endothelial markers. For example, a test cell or test sample may be further or additionally probed for the presence or expression of one or more of the following:
(i) CD44 (a cell-surface glycoprotein involved in cell-cell interactions, cell adhesion and migration)
(ii) Ecto-5′-nucleotidase (CD73);
(iii) Sushi domain-containing protein 2 (SUSD2);
(iv) Integrin, beta 1 subunit (CD29); and
(v) CD90 (Thy-1)

Wherein a test cell or test sample shown to express or contain an amount of one or more of the markers listed as markers (i)-(v) may contain an E-MSC, for example an ectopic E-MSC.

It should be noted that E-MSCs whether eutopic or ectopic, may be characterised by the absence, or very low amounts of, certain other molecules (for example certain cell surface markers). For example, an E-MSC may be characterised by low levels (or substantially no expression) of CD146, CD34, CD45, EPCAM and/or Tie2. In this regard, a test cell or test sample may be further or additionally probed for the presence or expression of one or more of the following:
(a) melanoma cell adhesion molecule (CD146);
(b) CD34;
(c) RTP Type C (receptor tyrosine phosphatase, CD45);
(d) Epithelial cell adhesion molecule EpCAM and
(e) TEK receptor tyrosine kinase (TIE2);
   wherein test cell or test sample shown to express or contain a low amount or substantially no amount of any one or more of the markers listed as markers (a)-(e), may contain an E-MSC, for example an ectopic E-MSC.

In all cases, the step of detecting, for example D₂, CD105 and/or CD140b (or some other molecule as detailed herein), either in a sample or on the surface of a cell, for example an E-MSC, may involve the use of antibodies with affinity and/or specificity for the relevant molecule. For example, the disclosed methods may use anti-dopamine receptor 2 antibodies, anti-CD105 antibodies and/or anti-CD146b antibodies.

Useful antibodies may be conjugated to or labelled with detectable moieties, for example optically delectable moieties. Examples of useful and detectable moieties may include, fluorescent, chemiluminescent and/or coloured particles-such as colloidal gold, alkaline phosphatase, horseradish peroxidase and the like. One of skill will appreciate that binding a labelled and (optically) detectable antibody to a particular molecule (for example a CD molecule or one of the abovementioned target molecules), allows expression of that molecule to be detected and/or quantified.

It should be noted that the term antibody includes any target molecule binding fragment thereof. Within the context of this disclosure, the term “target molecule” may include any of the mesenchymal, haematopoietic, endometriotic, epithelial and/or endothelial markers/molecules described above.

Immunological techniques such as ELISA, immunohistochemistry and FACS may be used to detect or visualise bound antibody and subsequently to report levels of target molecule expression. Moreover, the amount of antibody bound (and the corresponding amount of any detectable label present on the bound antibodies) may be used as a means by which the amount of any given target molecule may be quantified.

An application of the disclosed methods of detection is in the diagnosis of ectopic endometriosis. As stated, samples may be provided by, or obtained from a subject, and that sample then probed or analysed for the presence or absence of any of the target molecules described herein. Depending on the results, the sample may be identified as having been obtained from or provided by a subject who:
(i) is suffering from ectopic endometriosis;
(ii) is susceptible/predisposed to endometriosis;
(iii) is convalescing from or is between occurrences of ectopic endometriosis.

The precise result which determines what conclusions a user may draw about the origin of a sample may depend on the nature of the control against which the results are compared. For example, where the results are compared to a sample comprising eutopic E-MSCs, then a sample obtained from any of subject types (i)-(iii) above will report a relatively higher amount of D₂, and optionally a relatively higher amount of CD105 and a relatively lower amount of CD140b.

Any method of diagnosing ectopic endometriosis and/or a subject pre-disposed or susceptible thereto, may further comprise allocating a treatment to that subject. For example, a subject diagnosed as having, or being susceptible to ectopic endometriosis may be allocated or earmarked for, a quinagolide-based treatment regime. In another teaching, a method of diagnosis may include a step of administering quinagolide to a subject.

The identified differential expression of at least D₂ between ectopic E-MSCs (where expression is relatively high) and eutopic E-MSCs (where expression is relatively low) further lends itself to a method in which a subject's disease progression, recovery from disease and/or response to treatment may be monitored.

For example, test samples provided by a subject suffering from ectopic endometriosis may be characterised by relatively high amounts of ectopic E-MSCs (relative to, for example some sample of healthy (disease free) tissue or a sample containing only eutopic E-MSCs).

A relative high amount of ectopic E-MSCs may, in turn, result in a relatively high amount of D₂ in the test sample (again relative to some control amount derived, for example, from a sample of healthy (disease-free) tissue which lacks the D₂ or a sample which contains only eutopic E-MSCs). By monitoring the amount of D₂ present in a sample, it may be possible to determine whether or not a subject is recovering from disease and/or responding to treatment.

If a subject is beginning to recover from an episode of ectopic endometriosis and/or is responding to treatment (for example treatment with quinagolide) one would expect the amount of D₂ present in a sample provided by the subject to fall over time. A subject who is not recovering and/or not responding to treatment or in whom the disease is progressing and/or getting worse, may provide a sample in which the amount of D₂ remains constant or increases over time.

Accordingly, the disclosure provides a method of staging ectopic endometriosis in a subject or monitoring the progression of ectopic endometriosis in a subject. The phrases “staging ectopic endometriosis” and “monitoring the progression of ectopic endometriosis” may embrace the process of determining whether or not an incidence of ectopic endometriosis in a subject is active, progressing, regressing, resolving, improving, worsening and/or in remission.

As stated, methods of this type may be useful as they allow the user to determine whether or not a subject is responding to treatment and/or also to determine, at any given time point, the likelihood of the recurrence of an episode of ectopic endometriosis in a subject. In such cases the subject may, for example, be a subject who has had an operation or other procedure to remove ectopic endometrial lesions. After such procedures, the subject may generally be advised to adopt or resume a prophylactic treatment—for example a hormone-based treatment, for example a contraceptive medication. Medication of this type minimises the chances of disease re-occurrence. Where the subject would like to conceive, the advice may be to try conceiving as soon as possible after surgery and for a brief period while the disease is in remission or under control. However, recurrence is unpredictable and a subject would be advised to take or resume prophylactic medication sooner rather than later. A method of this disclosure, which method may probe samples for a level of D₂, may be used to stage the progression of ectopic endometriosis and make a determination as to whether or not (and/or when) an episode of ectopic endometriosis may re-occur and when prophylactic treatment options may need to resume. It should be noted that (as explained elsewhere) a patient recently subject to a surgical procedure to remove ectopic endometriosis lesions, may return samples with relatively low amounts of D₂ (indicative of low numbers of ectopic E-MSCs). In contrast, a subject in whom (for example, after surgery) an episode of ectopic endometriosis is about to re-occur or is re-occurring, the levels of D₂ in a sample may (relative to at least the levels of D₂ present in a sample obtained immediately after treatment or surgery) be higher and increasing over time and may approach the levels of D₂ characteristic of a sample obtained from a subject with (an active case of) ectopic endometriosis. The methods of the present disclosure may provide an indication of how likely ectopic endometriosis is to re-occur in a subject, thus informing the subsequent treatment options. In some cases, a method of this disclosure may extend the conception window after surgery/treatment for ectopic endometriosis and may allow a subject to avoid unnecessary treatments. A method of this type may require probing a sample (for example a sample of any of the types described herein) for the presence or absence of an amount of D₂ and comparing that amount to some control or reference value—for example a control or reference amount of D₂.

The control or reference amount of D₂ may be indicative of a known disease status—for example a resolved disease status, an improving disease status and/or an active disease status. In one teaching, the detected amount of D₂ may be compared against two or more control or reference amounts of D₂—for example, one control or reference amount indicative of an active disease status and one control or reference amount characteristic of healthy tissue or a healthy subject.

Where the control or reference amount of D₂ is indicative of a resolved or resolving episode of ectopic endometriosis and the detected amount of D₂ in the sample is relatively higher, the subject (from whom the sample has been obtained) may be suffering from ectopic endometriosis, may have a worsening case of ectopic endometriosis, may have lapsed into a further episode of ectopic endometriosis or may not be responding to treatment.

Where the control or reference amount of D₂ is indicative of an active episode of ectopic endometriosis and the detected amount of D₂ in the sample is relatively lower, the subject (from whom the sample has been obtained) may be recovering from an episode of ectopic endometriosis, may have an improving case of ectopic endometriosis, may have moved into remission from the disease or may be responding to treatment.

Any subject may provide a sample for use in the staging or monitoring methods described herein. For example, the subject may be:
a heathy subject—i.e. a subject who has not had or does not have ectopic endometriosis; or
a subject being treated for ectopic endometriosis; or
a subject recovering or convalescing from ectopic endometriosis;
a subject with ectopic endometriosis (i.e. with active ectopic endometriosis); or
a subject susceptible or predisposed to ectopic endometriosis.

The disclosed staging/monitoring methods (which rely on determining an amount of D₂ expression in a sample) may be supplemented with steps which determine an amount, a level of expression or the presence or absence of one or more other molecules/targets/markers. For example, a method of staging/monitoring may further comprise determining an amount of endoglin (CD105) in a, or the, sample. Additionally or alternatively, a method of staging/monitoring may further comprise determining an amount of platelet derived growth factor receptor beta (PDGFRβ or CD140b) in a, or the, sample.

Again, the precise result which determines what conclusions a user may draw about the stage or progression of ectopic endometriosis in a subject may depend on the control against which the results are compared. For example, where the results are compared to a sample comprising eutopic E-MSCs, then a sample provided by a subject with an active, non-treatment responding or worsening case of ectopic endometriosis, will report a relatively higher amount of endoglin (CD105) and/or a relatively lower amount of platelet derived growth factor receptor beta (PDGFRβ or CD140b). In contrast, a sample provided from a healthy subject, a subject recovering from ectopic endometriosis, in remission from ectopic endometriosis or successfully responding to treatment, may report either similar levels of endoglin (CD105) and/or platelet derived growth factor receptor beta (PDGFRβ or CD140b) (i.e. similar to the control levels) or improving levels (lowering levels of endoglin (CD105) and increasing levels of platelet derived growth factor receptor beta (PDGFRβ or CD140b).

A method of staging/monitoring ectopic endometriosis may further comprise steps in which the, or a, sample is further or additionally probed for the presence of one or more of the following:
(i) CD44 (a cell-surface glycoprotein involved in cell-cell interactions, cell adhesion and migration)
(ii) Ecto-5′-nucleotidase (CD73);
(iii) Sushi domain-containing protein 2 (SUSD2);
(iv) Integrin, beta 1 subunit (CD29); and
(v) CD90 (Thy-1)
   wherein a sample shown to contain an amount of one or more of the markers listed as markers (i)-(v) may contain an E-MSC, for example an ectopic E-MSC—the results of these assays may allow the user to confirm that the sample was of the correct type and that it contained E-MSC.

As stated, E-MSCs whether eutopic or ectopic, may be further characterised by the absence, or very low amounts of, certain other molecules (for example certain cell surface markers). For example, an E-MSC may be characterised by low levels (or substantially no expression) of CD146, CD34, CD45, EPCAM and/or Tie2. In this regard, in order to confirm that a sample provided for a staging or monitoring method of this disclosure, contains E-MSCs, the sample may be further or additionally probed for the presence or expression of one or more of the following:
(a) melanoma cell adhesion molecule (CD146);
(b) CD34;
(c) RTP Type C (receptor tyrosine phosphatase, CD45);
(d) Epithelial cell adhesion molecule EpCAM and
(e) TEK receptor tyrosine kinase (TIE2);
   wherein a sample shown to express or contain a low amount or substantially no amount of any one or more of the markers listed as markers (a)-(e), may contain an E-MSC, for example an ectopic E-MSC.

The staging or monitoring methods may exploit any of the antibodies described herein as a means to detect the expression, presence or absence of the various markers.

The disclosure further provides a kit for detecting ectopic endometrial mesenchymal stromal cells (E-MSCs), said kit comprising an anti-dopamine receptor 2 antibody. The kit may further comprise an anti-CD105 antibody and/or anti-CD140 antibody.

The kit may further comprise one or more antibodies selected from the following:
(i) An anti-CD44;
(ii) An anti-CD73;
(iii) An anti-CD146;
(iv) An anti-SUSD2;
(v) An anti-CD29;
(vi) An anti-CD90;
(vii) An anti-CD34;
(viii) An anti CD45;
(ix) An anti-EpCAM; and
(x) An anti-TIE2.

The antibodies contained within a kit of this disclosure may comprise or be conjugated, fused or bound to, a detectable label or moiety, for example an optically detectable label or moiety.

A kit of this disclosure may further comprise buffers, solutions, tools, receptacles for use in any of the methods described herein.

A kit of this disclosure may comprise, instructions for use.

### DETAILED DESCRIPTION

The disclosure will now be further described with reference to the following figures which show:

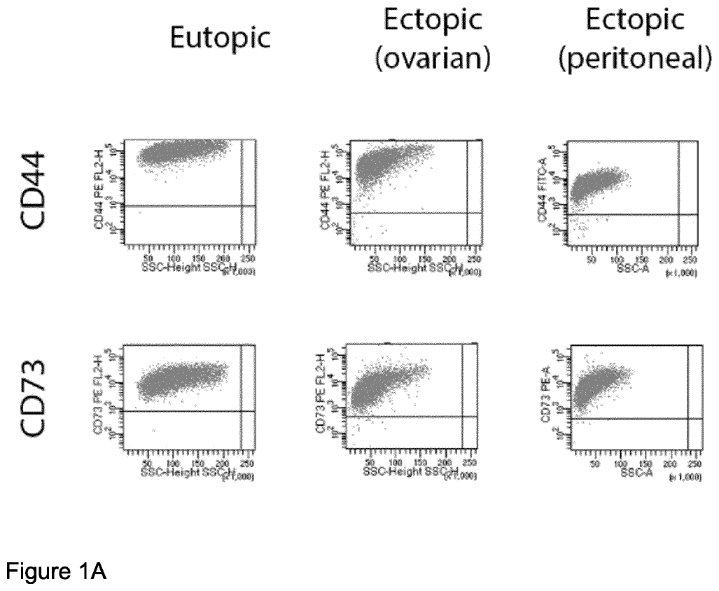

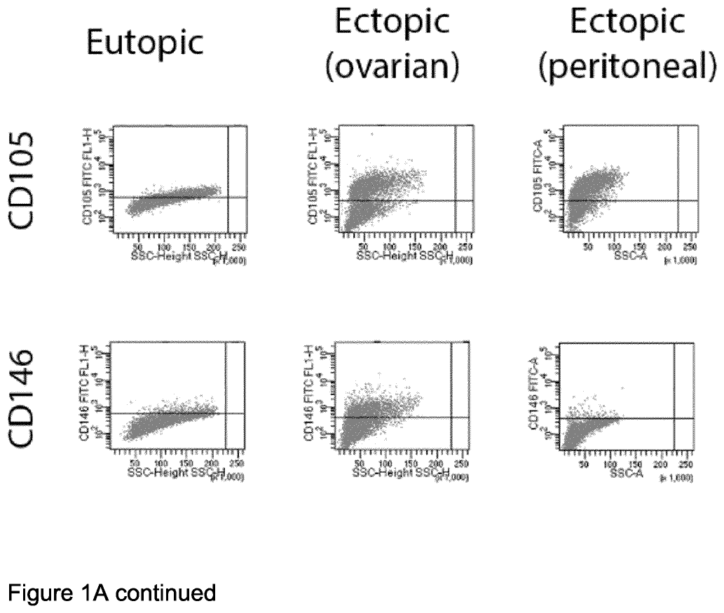

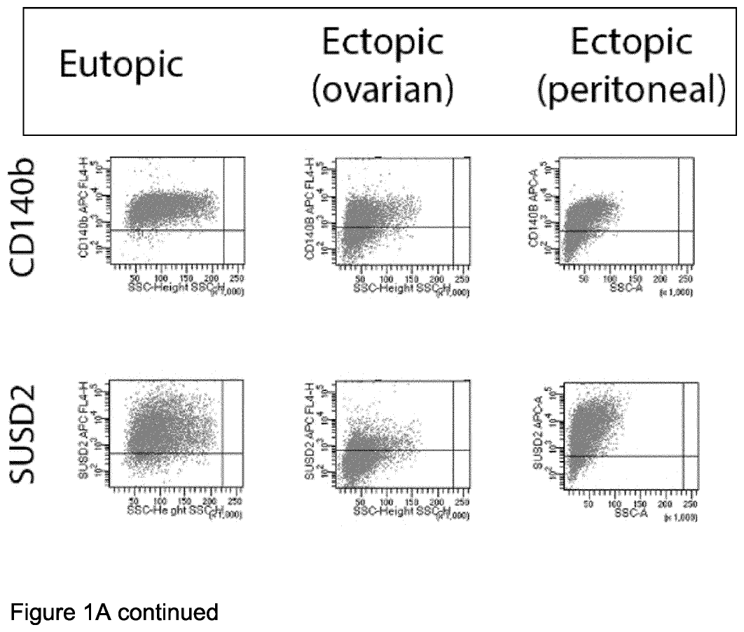

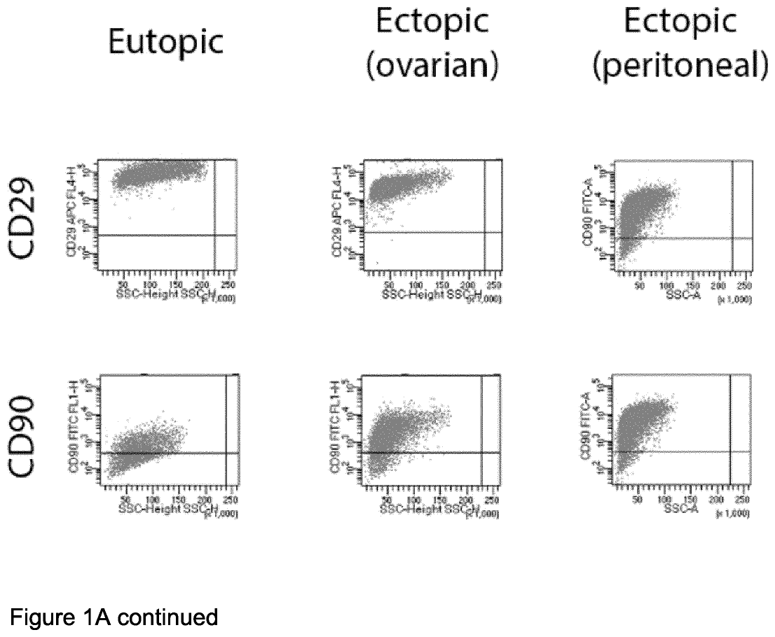

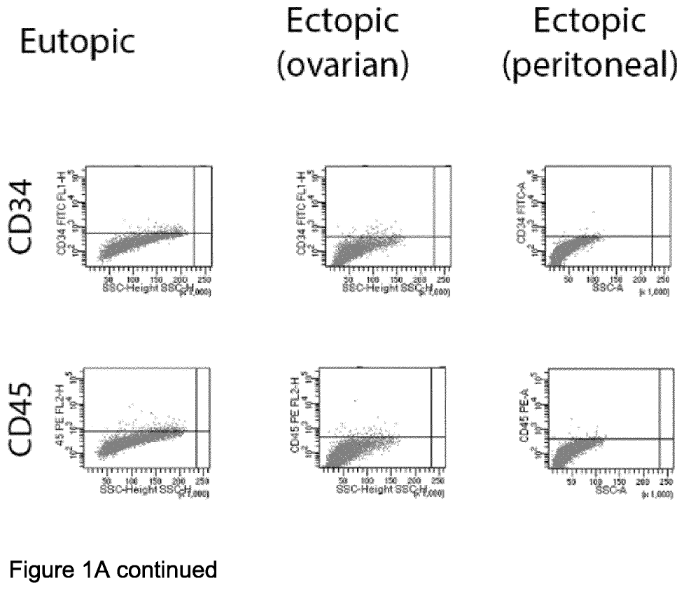

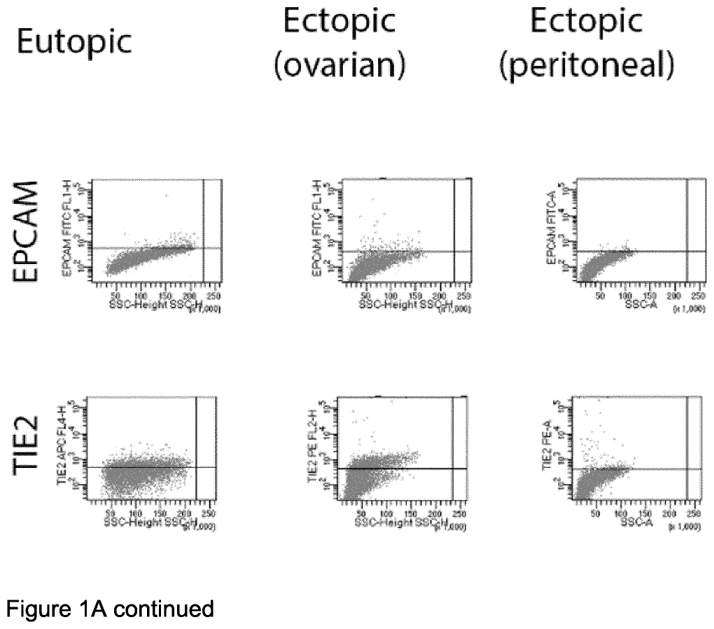

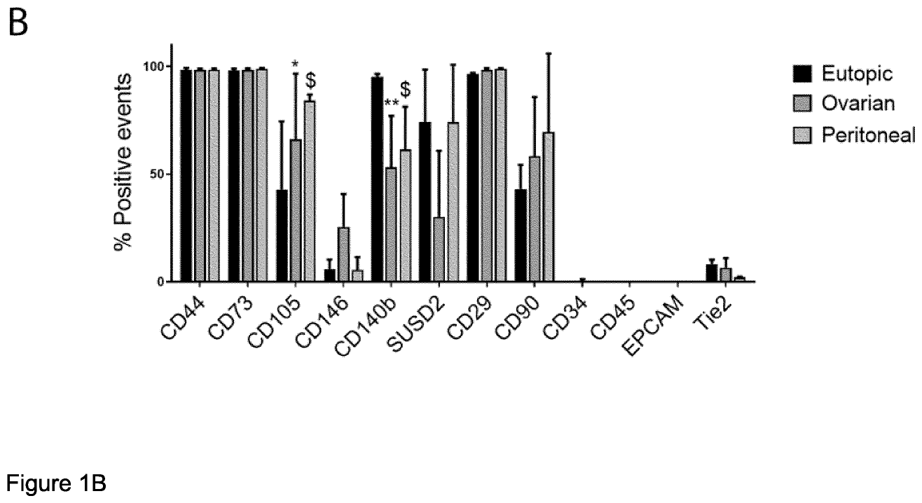

FIG. 1: Expression of mesenchymal, hematopoietic, endometriotic, epithelial and endothelial markers by human eutopic and ectopic E-MSCs. Representative FACS analysis (A) and quantification (B) of eutopic and ectopic (both ovarian and peritoneal) E-MSCs lines for the expression of mesenchymal (CD44, CD73, CD90, CD29, CD105, CD146), hematopoietic (CD45, CD34), endometriotic (SUSD2, CD140b), epithelial (EPCAM) and endothelial (TIE2) markers. Analyses were performed on every cell line used in the study between passage 1 and 2. Data are shown as mean±SD of all the tested lines: eutopic (N=3), ovarian (N=6) and peritoneal (N=3) ectopic E-MSCs. P: p-value; *=p<0.05, **=p<0.01 (ovarian vs eutopic); $=p<0.05 (peritoneal vs eutopic).

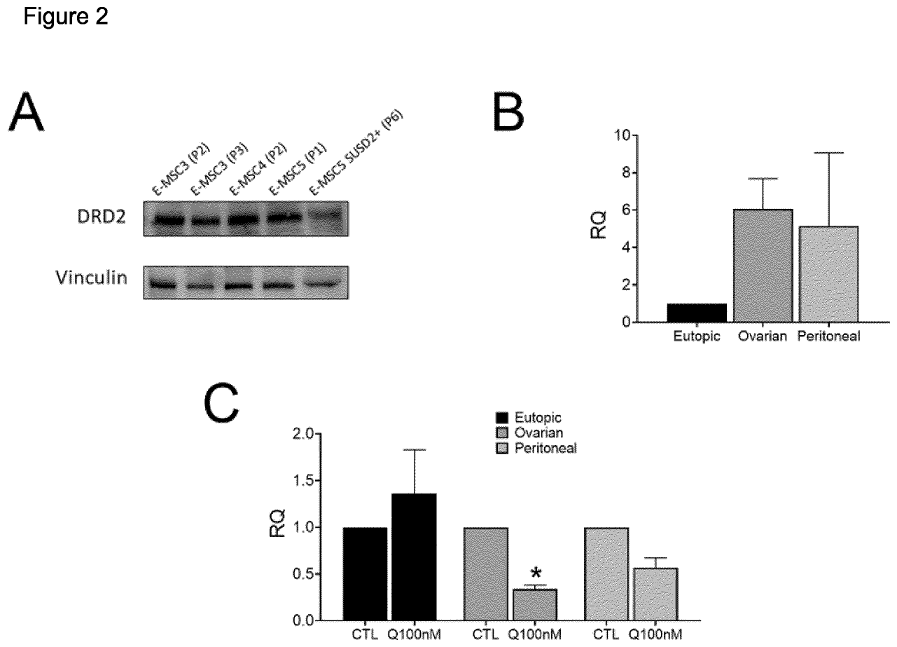

FIG. 2: Effect of quinagolide on D₂ expression in eutopic and ectopic EMSCs. A: Western blot analysis showing the presence of D₂ in EMSCs lines, at different passages, and in sorted SUSD2⁺ E-MSCs. B: Real Time PCR analysis showing the relative quantification (RQ) of D₂ mRNA expression by eutopic and ectopic EMSCs. Data are represented as mean±SD of three different eutopic or ectopic (ovarian and peritoneal) lines and normalized to GAPDH and to eutopic EMSCs. C: Real Time PCR analysis showing D₂ mRNA expression after 48 h of 100 nM quinagolide treatment (Q100) by eutopic and ectopic EMSCs. Data are represented as mean±SD of three different eutopic or ectopic (ovarian and peritoneal) lines and normalized to GAPDH and to untreated EMSCs (CTL). ANOVA was performed: *=P<0.05 vs CTL.

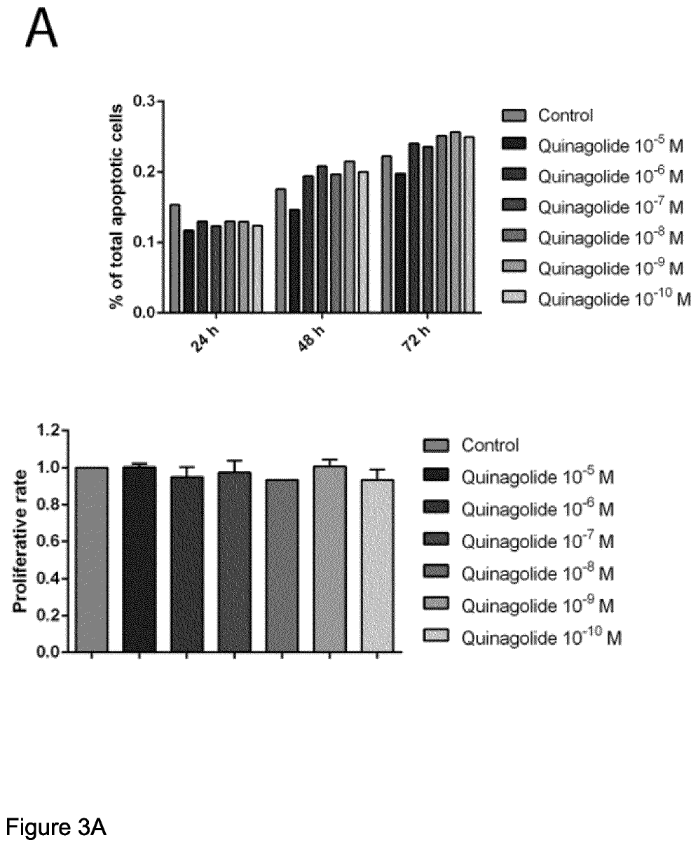

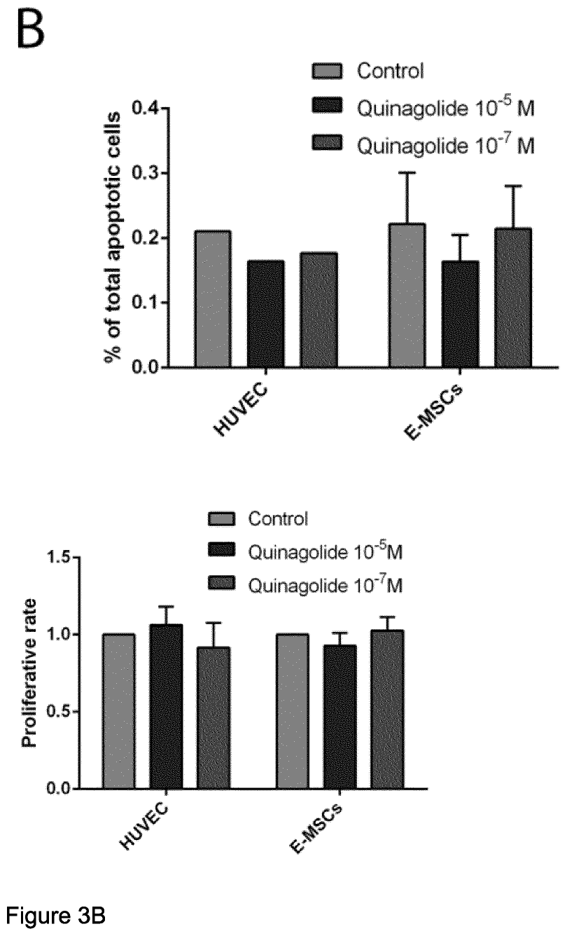

FIG. 3: Quinagolide effect on EMSCs apoptosis and proliferation. A: Quinagolide concentration-response curve on ectopic EMSCs in both apoptosis (N=1) and proliferation (N=2) assays. B: Effect of two selected quinagolide doses (10⁻⁵ and 10⁻⁷M) on HUVECs (N=1) and ectopic E-MSCs (N=3) apoptosis and proliferation assays (N=2). Data are represented as mean±SD of the indicated number of experiments and normalized to untreated cells (Control).

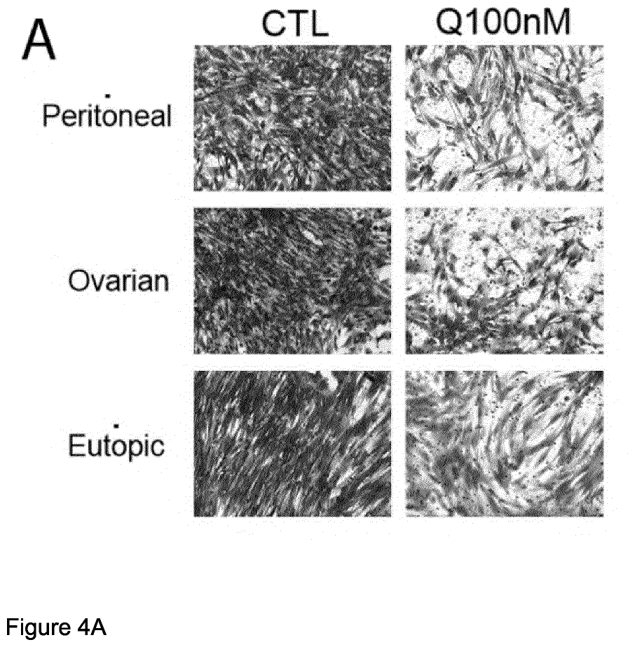

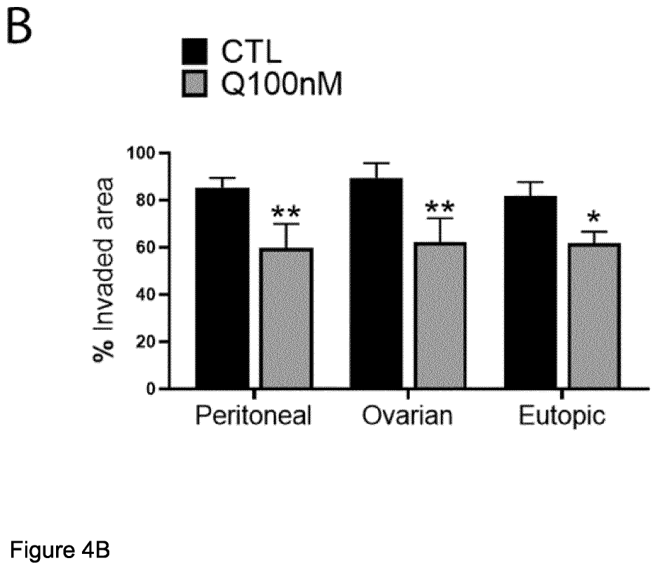

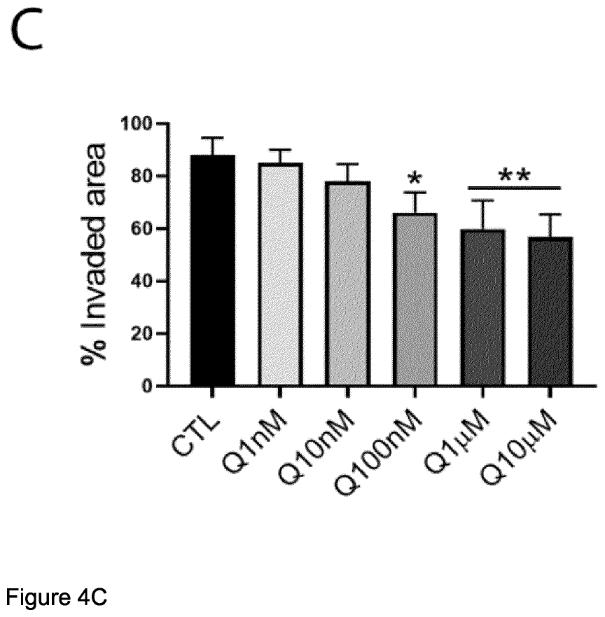

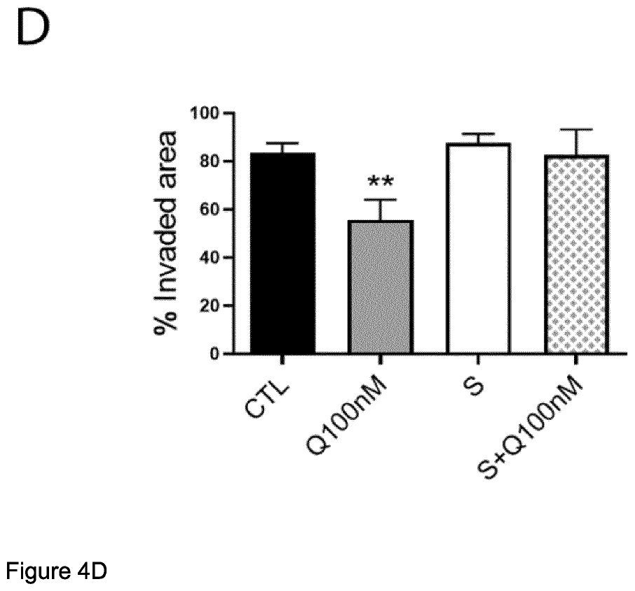

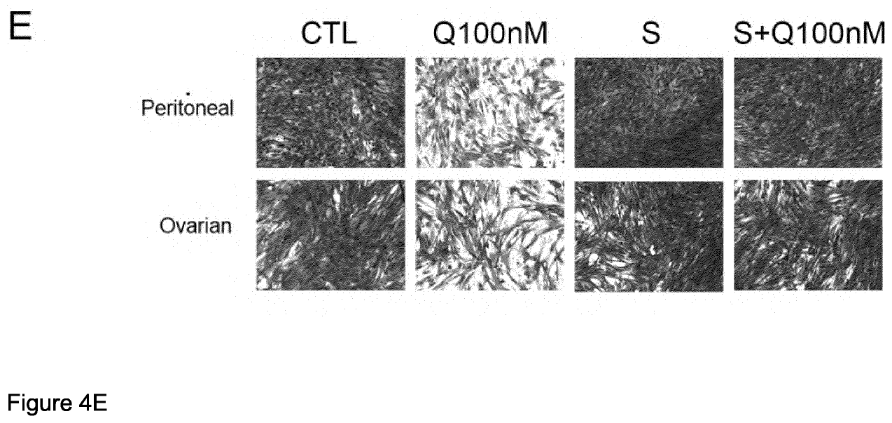

FIG. 4: Quinagolide effect on EMSCs invasion. A and B: Representative micrographs (A) and quantification (B) of quinagolide effect (100 nM) on eutopic and ectopic (both ovarian and peritoneal) EMSCs invasion (original magnification: ×100). C: Concentration response effect of quinagolide treated ectopic EMSCs invasion. D and E: Quantification (D) and representative micrographs (E) (original magnification: ×100) of invasion assays performed on ectopic EMSCs (both ovarian and peritoneal), treated with 100 nM quinagolide (Q100 nM), 5 μM spiperone(S) or a combination of quinagolide and spiperone (S+Q100 nM). All invasion data are represented as mean±SD of at least three independent experiments, performed on different EMSC lines, and normalized to untreated cells (CTL). One-way ANova was performed: *=p<0.05 and **=p<0.001 vs CTL.

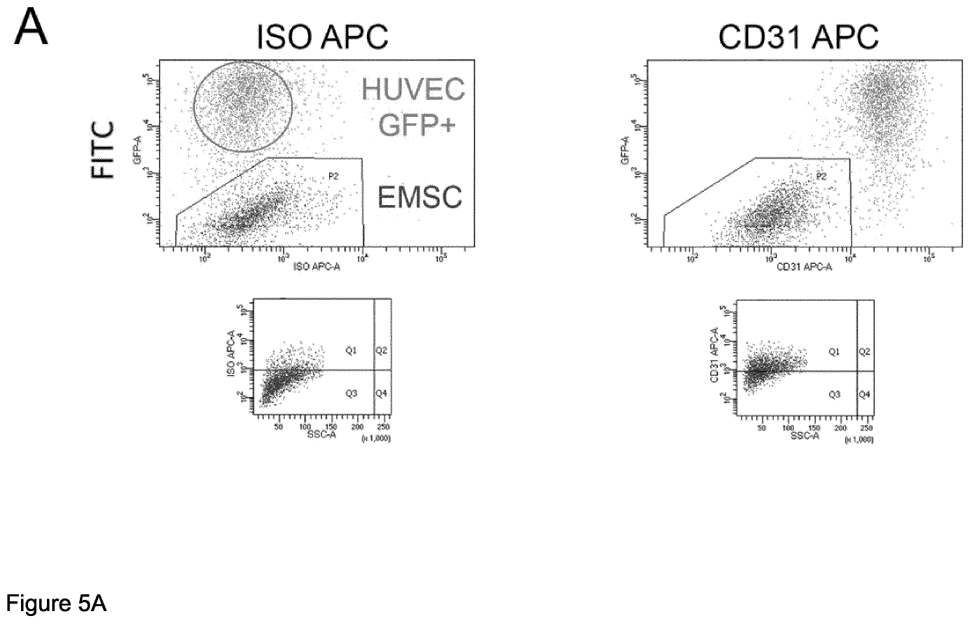

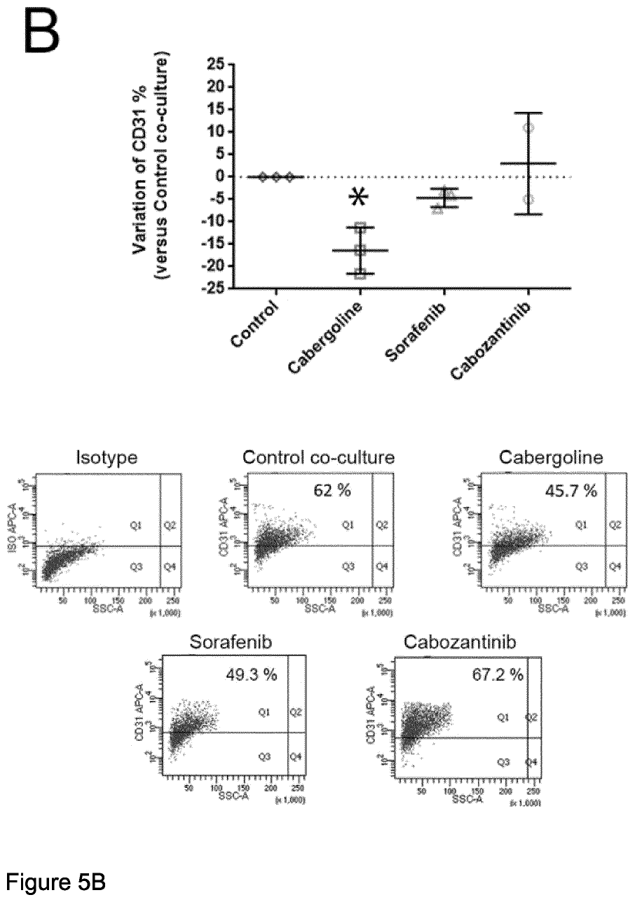

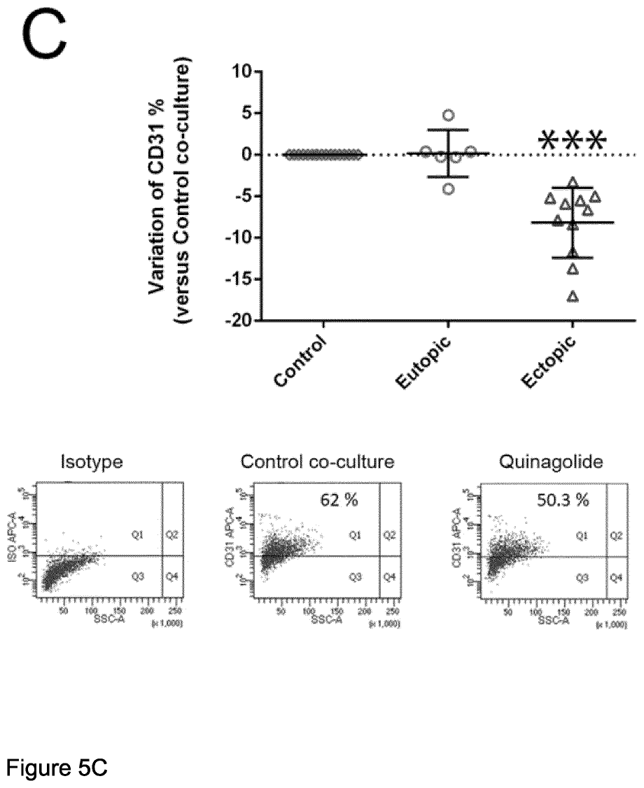

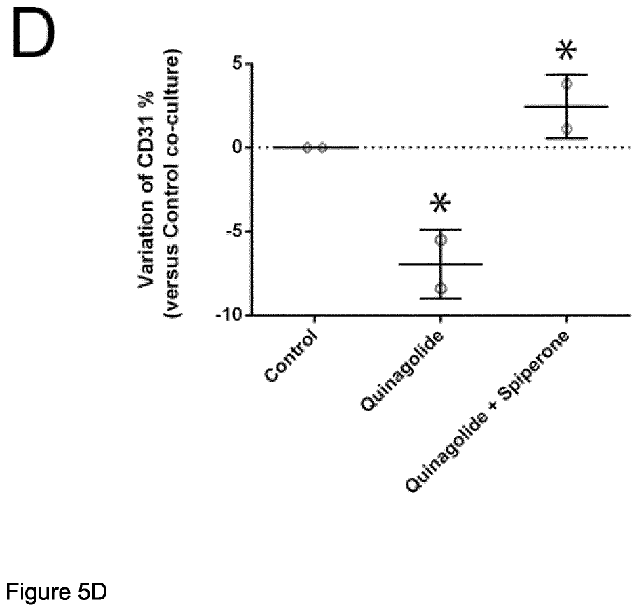

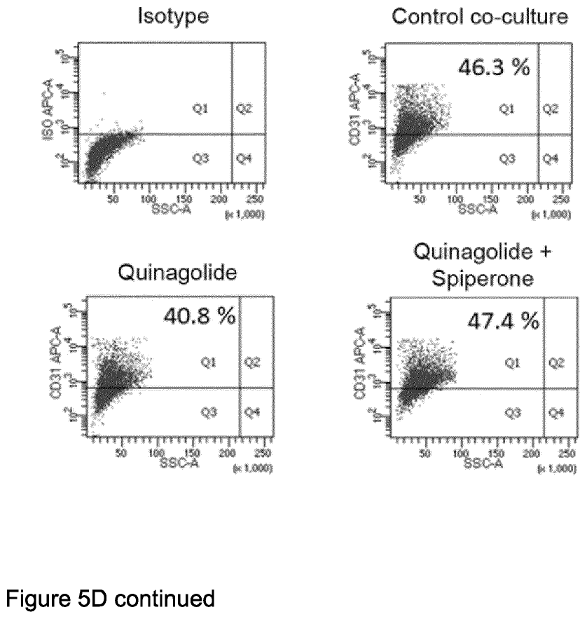

FIG. 5: Quinagolide effect on EMSCs endothelial differentiation. A. FACS gating-strategy for the analysis of CD31⁺ EMSCs after direct 48 h co-culture with HUVECs. EMSCs are gated as GFP negative population, and CD31 expression is evaluated on the described gated EMSCs after 48 h co-culture with HUVECs as percentage of CD31 APC positive events. B, C and D: Representative flow cytometry micrographs and quantification, expressed as percentage of variation respect to control co-culture, of the effect of 24 h treatment of EMSCs with 25 μM cabergoline, 1 μM sorafenib, 1 μM cabozantinib (B), 100 nM quinagolide (C) and the combination of 5 μM spiperone and 100 nM quinagolide (D) on EMSCs CD31 expression after 48 h of co-culture with HUVECs. Data are represented as mean±SD of at least three independent experiments, performed on different ectopic EMSC lines, and normalized to untreated co-culture. One-way ANova was performed: *=p<0.05 and ***=p<0.001 vs control.

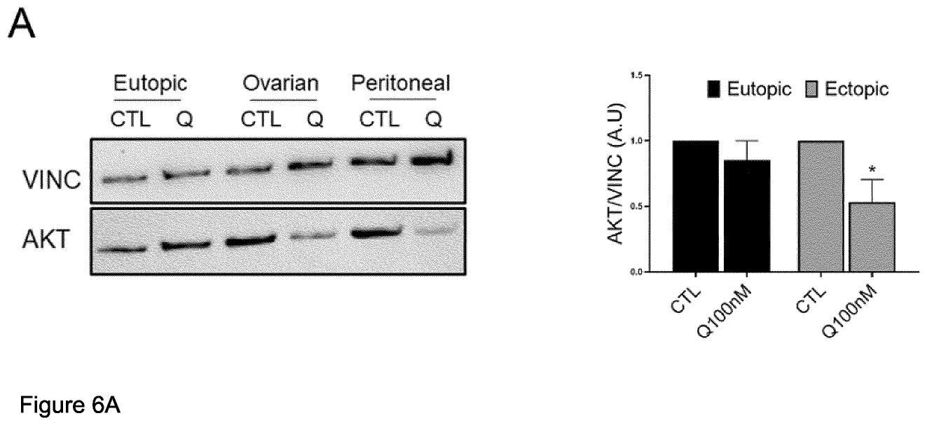

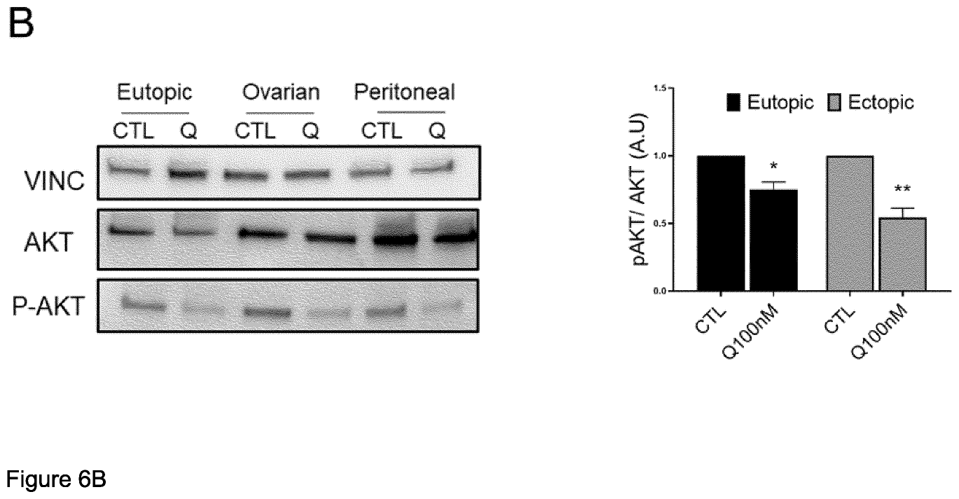

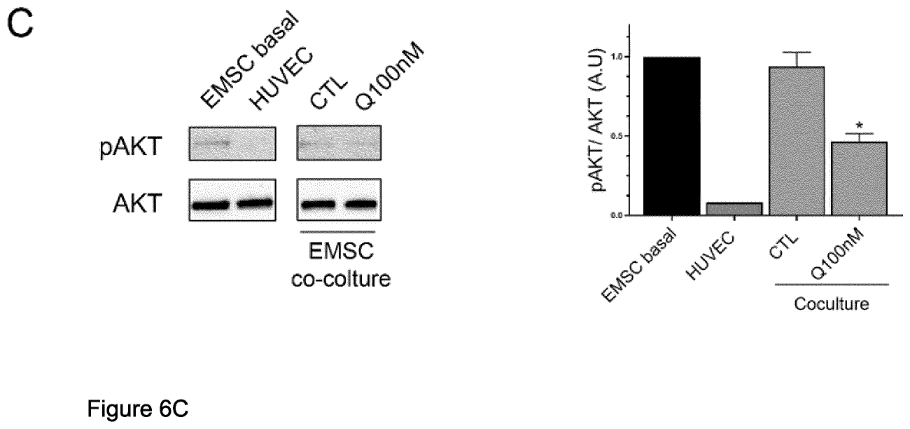

FIG. 6: Quinagolide effect on AKT activation. A: Representative western blot analysis and quantification of AKT levels in eutopic and ectopic EMSCs treated for 48 h with 100 nM quinagolide (Q), compared to untreated cells (CTL). B: Representative western blot analysis and quantification of P-AKT levels, normalized to AKT expression, in eutopic and ectopic EMSCs treated for 48 h with 100 nM quinagolide (Q), respect to untreated cells (CTL). C: Representative western blot analysis and quantification of P-AKT levels in HUVECs, ectopic EMSCs (EMSC basal), and in EMSCs after co-culture with HUVECs, treated or not for 24 h with 100 nM quinagolide (Q100 nM). Data are represented as mean±SD of at least two independent experiments, performed on different EMSC lines, and normalized to Vinculin and to untreated cells (CTL). One-way ANOVA was performed: *=p<0.05 and **=p<0.001 vs CTL.

### MATERIALS AND METHODS

### Patients

A total of 10 patients were enrolled in the present study for tissues collection and subsequent cell lines isolation. All patients provided preoperative written informed consent before receiving endometrial sampling or surgery for treatment of ovarian or/and peritoneal endometriosis in the Department of Surgical Sciences at the University of Turin, after approval by the Ethics Review Board of the Health and Science City of Torino, (Città della Salute e della Scienza di Torino).

### Endometriotic Specimen Collection and E-MSC Isolation

Three eutopic samples were collected by gently scraping the endometrium of control patients, used as controls, whereas the other nine ectopic samples were obtained by surgical biopsy of the inner wall of the ovarian or peritoneal endometrial tissue of endometriotic patients. whereas nine ectopic tissues were obtained by surgical biopsy of the inner wall of the ovarian cyst or of peritoneal lesions of endometriotic patients. In two patients both ovarian and peritoneal endometrial samples were collected since the patients presented the two different type of endometriosis. The tissues were immediately processed by dissection into small fragments in a sterile tissue culture dish using a sterile scalped blade in a laminar flow hood. The fragments were first enzymatically digested with 0.1% Type I Collagenase (Sigma-Aldrich, St. Louis, MO, USA) for 30 min in a 37° C. heater and, then they were mechanically disaggregated through 60-mm and 120-mm meshes. After two times 10 minute centrifugations at 1,500 g for washing, the pellet was resuspended in EBM plus supplement kit (Lonza, Basel, Switzerland) as described for E-MSC isolation (Moggio et al., 2012) and cells were seeded in T25 flasks. Dead cells were poured off 72 h later and cell clones were typically observed after 5-7 days, but medium was changed only after 7 days to guarantee cell attachment. Then, medium was recovered every 2-3 days and cells were passaged for the first time 10-14 days after plating, when confluence was reached. In the subsequent passages, cells were split two times per week. Twelve E-MSC lines were isolated (eutopic E-MSCs n=3, ectopic ovarian E-MSCs n=6, ectopic peritoneal E-MSCs n=3) and cultured for a maximum of 11 passages to evaluate their proliferative ability. All the experiments were performed between passages 1-7.

### Flow Cytometric Analysis

E-MSCs were characterized at passage 1 or 2 using FACS Celesta (BD Biosciences, San Jose, CA, USA). Cells were detached using a non-enzymatic cell dissociation solution (Sigma-Aldrich), centrifuged at 1200 rpm for 5 minutes and then resuspended in 100 μl of 0.1% Bovine Serum Albumin

(BSA)-Phosphate Buffered Saline (PBS) (Sigma). For each staining, 100,000 cells were incubated for 20 minutes at 4° C. with FITC, APC or PE-conjugated antibodies against: CD29, CD44, CD73, CD90 (BD Bioscences, Franklin Lakes, NJ, USA); CD31, CD34, CD105, CD140b, CD146, TEK receptor tyrosine kinase (Tie2), Sushi domain-containing protein 2 (SUSD2) (Miltenyi Biotech, Bergisch Gladbach, Germany); CD45 (AbD Serotec, Raleigh, NC, USA), or epithelial cell adhesion molecule (EPCAM) (BioLegend, San Diego, CA, USA). Labelled cells were washed by centrifugation and final pellet was resuspended in 200 μl of 0.1% BSA-PBS before cytofluorimetric analysis. Isotype (Miltenyi Biotec) was used as negative control.

### Protein Extraction and Western Blot

Cell pellets were lysed at 4° C. for 15 minutes in RIPA buffer supplemented with protease and phosphatase inhibitors cocktail and PMSF (Sigma-Aldrich). Proteins were quantified using Bradford solution following the manufacturer procedures (Bio-Rad Inc., Berkely, CA, USA) and aliquots of cell lysates containing 50 μg of proteins were run on 4-12% Mini-Protean TGX Stain-Free Gels (Bio-Rad) under reducing conditions and transferred onto PVDF membrane filters using the iBLOT2 system (Life Technologies). Each membrane was immersed in blocking solution (5% milk powder in PBS (Sigma)) for 1 h before overnight incubation with the following primary antibodies: anti-D₂, anti-Vinculin 1:8000 (Sigma-Aldrich), anti-AKT and anti-P-AKT (both from Cell Signalling). After rinsing in wash buffer (0.1% Tween in PBS) horseradish peroxidase-conjugated secondary antibodies (Thermo Scientific) were used for 1 h at 1:3000 dilutions. Membranes were finally washed and incubated with ECL chemiluminescence reagent (Bio-Rad) in a Chemidoc machine (Bio-Rad).

### Drugs and Reagents

Quinagolide powder (provided by Ferring Pharmaceuticals) was stored at 4° C. and resuspended in dimethylsulphoxide (DMSO) to a stock solution of 1 mM immediately before use. Spiperone powder (Sigma-Aldrich) was resuspended in water to a stock concentration of 1 mM and stored at −20° C. Cabergoline powder (Sigma-Aldrich) was dissolved in DMSO to a stock concentration of 25 mM and stored at −20° C. Sorafenib and cabozantinib (Sigma-Aldrich) were resuspended in DMSO to a final concentration of 10 mM and according to the manufacturer's instructions, and stored at −20° C. and −80° C., respectively. Quinagolide, spiperone and cabergoline were diluted 1:100 (final concentration 100 nM), 1:1000 (final concentration 5 μM) and 1:1000 (final concentration 25 μM) respectively. Quinagolide, cabergoline, sorafenib and cabozantinib and were administered for 24 hours during co-culture experiments, while spiperone was added to culture medium 1 h before quinagolide treatment. For invasion assays, E-MSCs were treated with spiperone 1 h before the cell detachment and quinagolide was added only when cells were plated on Matrigel.

### RNA Isolation and Real Time PCR

Trizol Reagent (Ambion) was used to isolate total RNA of different cell preparations, according to the manufacturer's protocol. RNA was then quantified spectrophotometrically using Nanodrop ND-1000. Quantitative real-time PCR was performed for gene expression analysis. Briefly, using the HighCapacity cDNA Reverse Transcription Kit (Applied Biosystems), first-strand cDNA was produced from 200 ng of total RNA. Real-time PCR experiments were then performed in a 20-μl reaction mixture containing 5 ng of cDNA template, the sequence-specific oligonucleotide primers (all purchased from MWG-Biotech) and the Power SYBR Green PCR Master Mix (Applied Biosystems). GAPDH mRNA was used to normalize RNA inputs. Fold change expression respect to control was calculated for all samples.

### Cell Proliferation Assay

Cells were plated in growth medium at a concentration of 2,500 HUVECs/well and 3,000 E-MSCs/well in a 96-multiwell plate. The day after quinagolide was added to the growth medium at different concentrations after 24 hours. Deoxyribonucleic acid synthesis was detected as incorporation of 5-bromo-2-deoxyuridine (BrdU) into the cellular DNA after 48 hours from cell plaiting, using an enzyme-linked assay kit (Chemicon). Untreated cells were used as control. Data are expressed as the mean±standard deviation (SD) of the media of absorbance of at least three different experiments and normalized to control.

### Apoptosis

Annexin V assays were performed using the Muse™ Annexin V & Dead Cell Kit (Millipore), according to the manufacturer's recommendations. Briefly, 20×10³ cells were plated and after 24 h treated with different concentrations of quinagolide. After 24, 48 and 72 hours, cells were detached and resuspended in Muse™ Annexin V & Dead Cell Kit and the percentage of apoptotic cells (Annexin V+) was measured. Data are expressed as the mean±standard deviation (SD) of the media of absorbance of at least three different experiments and normalized to control.

### e Assay

E-MSCs were seeded in triplicate in Matrigel-precoated (100 μg Matrigel/transwell) 8-um pore transwells at a concentration of 50,000 cells per well in 200 μl of RPMI 2% FCS with/without quinagolide at the indicated concentration. To test the D₂ antagonist effect, E-MSCs were pre-treated with spiperone (5 μM) for 1 h at 37° C. before cell detachment. After 48 hours, invaded E-MSCs on the bottom side of the transwell were fixed with methanol and stained with crystal violet. At least five pictures per transwell were acquired (original magnification: 100×), and the percentage of transwell area covered by invaded EMSCs was quantitatively measured by ImageJ software.

### HUVEC Purification and Generation of GFP Positive Cells

HUVECs derived from the umbilical vein vascular wall were plated on fibronectin-coated flasks and grown in endothelial cell basal medium with an EGM-MV kit (Lonza; containing epidermal growth factor, hydrocortisone, bovine brain extract) and 10% fetal calf serum in 37° C. and 5% CO₂ atmosphere incubator. Cells were transduced with lentiviral particles containing pGIPZ lentiviral vector (Open Biosystems, Lafayette, CO, USA) expressing green fluorescent protein (GFP). In particular, 293T cells were first transfected with pGIPZ construct adopting the ViralPower Packaging Mix (Life Technologies) and then the lentiviral stock was titered. HUVEC transduction was performed at the first passages and at 70% cell confluence following the manufacturer's instructions. After Puromycin (ThermoFisher, Waltham, MA, USA) (1000 ng/ml) selection, antibiotic-resistant HUVECs were expanded. Finally, FACS analysis was performed to evaluate the expression of endothelial markers and GFP+>98%.

### Indirect Co-Culture Assembly

An indirect co-culture assembly was obtained plaiting HUVECs and E-MSCs at a ratio of 1:1 (1.5×10⁴/cell line) in E-MSC medium in T25 and maintaining the co-culture for 48 h in 37° C. and 5% CO₂ atmosphere incubator. HUVECs and E-MSCs cultured alone were used as control for each experiment.

### Statistics

Data are shown as mean±SD and at least three replicates were performed for each experiment. Two-tail Student's t test was used for analysis when two groups of data were compared, while 2 way ANOVA with Dunnett's multiple comparison test was applied when comparing more than two groups of data. All statistical analyses were done with GraphPad Prism software version 6.0 (GraphPad Software, Inc.). P values of <0.05 were considered significant.

### Results

### Generation and Characterization of Eutopic and Ectopic E-MSC Lines

A cohort of ten patients was enrolled for the study, including control (N=3), ovarian (N=6) and peritoneal (N=3) endometriosis. The demographic and clinical aspects of the patient population are summarized in Table 1.

TABLE 1
Demographic and clinical characteristics of patients enrolled in the study.

| | | | | Average | |
|---|---|---|---|---|---|
| | | | | menstrual | |
| Patient | | Age | Previous | cycle length | Other |
| # | Endometrial samples | (years) | pregnancies | (days) | diseases |
| 1 | Eutopic | 37 | 1 | 28-30 | No |
| 2 | Eutopic | 40 | 2 | 28 | No |
| 3 | Eutopic | 38 | 2 | 29 | No |
| 4 | Ectopic (ovarian) | 42 | 1 | 27 | No |
| 5 | Ectopic (ovarian) | 46 | 2 | 30 | No |
| 6 | Ectopic (ovarian) | 42 | 3 | 28 | No |
| 7 | Ectopic (peritoneal | 31 | 2 | 28 | No |
| | and ovarian) | | | | |
| 8 | Ectopic (ovarian) | 38 | 0 | 31 | No |
| 9 | Ectopic (peritoneal | 36 | 0 | 27 | No |
| | and ovarian) | | | | |
| 10 | Ectopic (peritoneal) | 40 | 1 | 31 | No |

In particular, stromal cells from eutopic and ectopic tissues were isolated, as reported in detail in Materials and Methods, and cultured in EBM. After seven days, culture medium was refreshed, allowing the removal of dead and/or unselected cells and promoting the clonal growth of E-MSCs. Generated cell lines were analysed for their fibroblastic phenotype, adherence to plastic, and surface marker expression (Table 2 and FIGS. 1A and B). FACS analysis confirmed the mesenchymal phenotype of all E-MSC lines isolated (Moggio et al., 2012). In particular, expression of mesenchymal markers CD44, CD73, CD90 and CD29 was similar in eutopic and ectopic E-MSCs with only a statistically significant increase of CD105 in both ovarian and peritoneal ectopic E-MSCs compared to eutopic ones. Moreover, cell contamination was excluded by the lack of the epithelial marker EPCAM and the endothelial/hemopoietic markers CD34, CD45 and Tie2. All E-MSC lines were positive for specific endometriotic mesenchymal stem cell markers SUSD2 and PDGFRb (CD140b), with lower expression by ectopic E-MSCs in respect to eutopic ones suggesting that, as reported (Bianco et al., 2013; Gargett et al., 2016), E-MSCs represent a heterogenic population of mesenchymal stem cells and stromal fibroblast, sharing a number of markers and functions. In selected experiments, E-MSC lines were SUSD2 sorted to possibly enrich for the E-MSCs in respect to the stromal cells. By FACS analysis, the resulting SUSD2+E-MSC lines showed the same phenotypic profile compared to original E-MSCs (not shown) and SUSD2 expression returned to basal level after 1 culture passage. This observation confirms the idea that E-MSCs spontaneously differentiate into fibroblasts, and that E-MSC and fibroblast populations represent a continuum, and share characteristics and several functions (Barragan et al., 2016).

### Quinagolide Effect on the Invasion Potential of E-MSCs

E-MSC lines were used to evaluate the effect of quinagolide, a D₂ agonist, on E-MSC functional properties. D₂ agonists can inhibit VEGF-induced VEGFR-2 activity, by promoting D₂-VEGFR-2 cell surface association and VEGFR-2 dephosphorylation (Basu et al, 2001, Sinha et al., 2009). Therefore, we first evaluated the expression of both the quinagolide receptor D₂ and of VEGFR-2 (Basu et al, 2001; Sinha et al., 2009) on E-MSCs (FIG. 2). Quinagolide receptor D₂ was expressed by all E-MSC lines regardless of the passage number or the SUSD2 enrichment (FIG. 2A). Moreover, at the mRNA level, D₂ expression was significantly increased in ectopic lines (FIG. 2B). Quinagolide treatment reduced D₂ mRNA expression in ectopic lines, suggesting an effect on receptor downregulation (FIG. 2C). No expression of VEGFR2 was observed in E-MSCs (not shown), as previously reported (Canosa et al., 2017).

A concentration response curve showed lack of quinagolide effect on E-MSC proliferation or apoptosis (FIG. 3). As shown in FIG. 3A, different concentrations of quinagolide did not increase apoptosis of E-MSCs after 24, 48 and 72 hours. Moreover, quinagolide treatment did not affect proliferation of E-MSCs after 24 hours (FIG. 3A). Similarly, HUVEC cells, used as a control, did not show alteration in apoptosis and proliferation after 24 hours of quinagolide treatment (FIG. 3B). Based on these results, 100 nM of quinagolide was chosen for further experiments.

The effect of quinagolide on E-MSCs migration and invasiveness, a relevant feature in endometriosis (Kao et al), was evaluated using an invasion assay. As shown in FIG. 4A, eutopic and ectopic E-MSCs were able to invade Matrigel after 48 hours in culture. Interestingly, invasion of both eutopic and ectopic E-MSC lines was significantly reduced after 48 h quinagolide treatment (FIG. 4B), in a dose-dependent manner (FIG. 4C). Moreover, the quinagolide effect was completely reverted by pre-treatment with the D₂ antagonist spiperone (FIG. 4D), indicating that the observed anti-invasive effect of quinagolide was D₂ dependent.

### Quinagolide Effect on the Endothelial Differentiation of E-MSCs

Considering the reported ability of E-MSCs to differentiate into endothelial cells (Masuda et al., 2012; Canosa et al., 2017), the effect of quinagolide on a reported E-MSC-HUVEC co-culture model was tested, testing its in vitro endometriosis angiogenic potential.

Using this model, after 48 h direct co-culture of E-MSCs and HUVECs, CD31 expression was acquired by E-MSCs, confirming the influence of HUVECs in the differentiation potential of E-MSCs into endothelial cells (FIG. 5). The use of HUVECs traced by GFP expression (>98% in all experiments) allowed the CD31+/GFP+ HUVECs and the high CD31+/GFP− E-MSC population that results after co-culture to be easily separated by a selective FACS gating strategy (FIG. 5A), as reported (Canosa et al., 2017).

After 24 hours of direct co-culture assembly, cells were then treated with quinagolide and incubated for 24 hours before analysis. As cabergoline treatment (25 μM) was previously described to decrease the E-MSC's angiogenic potential, this drug was used as positive control (Canosa et al., 2017) (FIG. 5B). The typical increase in the percentage of CD31 expressing E-MSCs evaluated after co-culture was significantly reduced by 24-h quinagolide treatment in ectopic E-MSCs, while no significant alteration was measured in eutopic ones (FIG. 5C).

To confirm that the effect observed was due to the quinagolide treatment through its D₂, direct co-cultures were treated with the specific D₂ antagonist spiperone one hour before quinagolide treatment. As shown in FIG. 5D, the quinagolide effect was blocked by spiperone pre-treatment with no reduction in the percentage of CD31 expressing E-MSCs confirming that the effect observed on E-MSCs was D₂-mediated. Sorafenib and cabozantinib, anti-angiogenic tyrosine kinase inhibitors, had no effect on endothelial differentiation (FIG. 5D), as reported (Canosa et al., 2017), excluding the involvement of VEGFR-2 in this process. Moreover, quinagolide did not reduce the levels of VEGF in the coculture model.

### Molecular Mechanisms Related to Quinagolide Effect

In order to explain the molecular mechanisms at the basis of quinagolide's effect on E-MSCs, the AKT pathway was analysed after quinagolide treatment (FIG. 6). Quinagolide treatment reduced total AKT levels in ectopic EMSCs (FIG. 6A). A significant decrease in AKT phosphorylation in both eutopic and ectopic cell lines treated with quinagolide for 24 hours was observed (FIG. 6B). Moreover, quinagolide reduced the AKT phosphorylation when added to E-MSC-HUVEC cocultures (FIG. 6C). These data suggest the effect of D₂ agonists on AKT signaling, as already reported (Beaulieu et al., 2007). The effect on both AKT levels and on phosphorylation were more evident on ectopic E-MSC lines, in respect to eutopic ones, possibly in accordance with the increased expression of D₂ on these lines.

### Discussion

E-MSCs are postulated to play a critical role in the pathogenesis of endometriosis contributing in the establishment and progression of ectopic lesions supporting the vascularization and growth of the endometrial stromal tissue (Gargett et al., 2014). In the present study, it was demonstrated that a D₂ antagonist, quinagolide, inhibited the invasive properties of E-MSCs, and limited their endothelial differentiation in an endothelial co-culture model of angiogenesis.

Quinagolide is a non-ergot-derived D₂ agonist (Schade et al., 2007), described to be a safe and well-tolerated drug in the long-term prolactinoma treatment without severe side effects and several advantages when compared to other dopamine agonists (Schultz et al., 2000; Barlier et al., 2006). Comparison of the dopamine D₂ binding properties of different agonists (quinagolide, bromocriptine, pergolide and cabergoline) indicated quinagolide as the most potent D₂ agonist, with EC50 at 0.058 nM (Igbokwe et al, 2009). The first pilot study evaluating the possible use of quinagolide for endometriosis treatment involved patients simultaneously suffering from severe endometriosis and hyperprolactinemia (Gomez et al, 2011). Quinagolide treatment reduced the size of endometriotic lesions, possibly by acting through VEGFR-2 downregulation (Gomez et al, 2011). At present, the effect of quinagolide in endometriosis is under investigation in phase 2 clinical trials (NCT03749109, NCT03692403).

In this study, aiming at evaluating a pivotal effect of quinagolide on E-MSCs, D₂ expression in E-MSCs isolated from both eutopic and ectopic (peritoneal and ovarian) endometrial lesions was confirmed. Interestingly, D₂ levels appeared to be higher in ectopic E-MSCs. On the cell surface, D₂ may co-localize with VEGFR-2 (Sinha et al., 2009), and its activation may consequently limit VEGFR-2 phosphorylation and promote its endocytosis in endothelial cells. However, the lack of VEGFR-2 on E-MSCs may suggest that quinagolide's effect does not involve VEGFR-2. It did not show any impact on proliferation and apoptosis, whereas a dose dependent activity was observed on invasion inhibition, suggesting a possible therapeutic use in the reduction of endometriosis spread outside the uterine cavity. These results are consistent with the previously reported inhibitory effects of dopamine agonists on cancer cells and skin mesenchymal stem cell migration (Wang X, et al. 2019, Shome et al, 2012).

In addition, quinagolide was able to inhibit E-MSCs endothelial differentiation. A model of E-MSCs differentiation with activation of a number of endothelial genes when cocultured with endothelial cells was previously reported (Canosa et al, 2017). Herein, it was observed that quinagolide was able to reduce E-MSC differentiation, evaluated as the acquisition of the endothelial marker CD31. Importantly, quinagolide's effect was more prominent on ectopic rather than eutopic E-MSCs when added to the co-culture. This could be related to the increased expression of D₂ on ectopic E-MSCs. However, the absence of a pure mesenchymal stem population may underestimate the process of endothelial differentiation.

The inhibitory effect of spiperone, a selective Deantagonist, confirmed that the observed anti-invasive and anti-angiogenic effects of quinagolide were dependent from D₂ activation. Moreover, the observed effect was independent from the inhibition of VEGF release. Indeed, this model was independent of soluble factor release, and was previously shown to require cell contact (Canosa et al, 2017). Accordingly, quinagolide did not affect VEGF release. Moreover, sunitinib and cabozantinib, tyrosine kinase inhibitors blocking activation and signalling of growth factor receptors, (Patyna et al., 2008) including VEGFR-2, did not affect E-MSC endothelial differentiation.

Focusing on putative VEGFR-2 independent signalling pathways downstream of dopamine receptors, AKT activity was evaluated, previously reported as modulated by direct receptor activation (Beaulieu et al., 2007). Previous studies have convincingly shown that the AKT pathway mediates dopaminergic activities, and that manipulations of the AKT/GSK3 pathway results in significant alterations in dopamine-related functions and behaviours (Beaulieu et al., 2011). In the brain in particular, activation of D₂ may lead to a beta-arrestin mediated deactivation of AKT (Beaulieu et al., 2011) and decrease its phosphorylation, leading to a reduction of AKT activity (Han et al., 2019). A specific D₂ activation was also able to reduce the migration of skin MSCs to the wound beds by suppressing AKT phosphorylation (Shome et al, 2012). It was also found that quinagolide treatment of E-MSCs or of E-MSC/HUVEC co-cultures decreased AKT phosphorylation. Moreover, beside phosphorylation, AKT protein levels were reduced. Interestingly enough, ectopic E-MSC lines showed a better response to quinagolide in terms of AKT downregulation and deactivation, in accordance with the differential presence of D₂ and with the functional effect on the different E-MSC lines. These results confirmed the differential proliferation, migration, and angiogenic ability of ectopic E-MSCs reported in respect to eutopic E-MSCs from the same patient or from healthy patients (Moggio et al, 2012, Liu et al., 2020). The different D₂ expression and behavior of E-MSCs might be due to selection and/or epigenetic modulation of the extrauterine microenvironment found in ectopic sites, as reported for cancer lesions (Burney et al., 2012).

In conclusion, the effect of a D₂ agonist, quinagolide on E-MSC lines is reported for the first time, showing its effect on reduction of invasion and endothelial differentiation trough the Akt signaling pathway. Together with the reported effects on endometrial and endothelial cells, the observed prominent inhibitory effect of quinagolide on E-MSC ectopic cell lines, further support the rationale for use of this drug in endometriosis treatment.

### REFERENCES

1. Akyol A, Kavak E, Akyol H, Pala Ş, Gürsu F. The Non-Ergot Derived Dopamine Agonist Quinagolide as an Anti-Endometriotic Agent. Gynecol Obstet Invest. 2017; 82 (6): 527-532. doi: 10.1159/000452796. Epub 2016 Nov. 25. PMID: 27883998.
2. Barlier A, Jaquet P. Quinagolide—a valuable treatment option for hyperprolactinaemia. Eur J Endocrinol. 2006 February; 154 (2): 187-95. doi: 10.1530/eje.1.02075. PMID: 16452531.
3. Barragan F, Irwin J C, Balayan S, Erikson D W, Chen J C, Houshdaran S, Piltonen T T, Spitzer T L, George A, Rabban J T et al. Human endometrial fibroblasts derived from mesenchymal progenitors inherit progesterone resistance and acquire an inflammatory phenotype in the endometrial niche in endometriosis. Biol Reprod 2016; 94:118.
4. Basu S, Nagy J A, Pal S, Vasile E, Eckelhoefer I A, Bliss V S, Manseau E J, Dasgupta P S, Dvorak H F, Mukhopadhyay D. The neurotransmitter dopamine inhibits angiogenesis induced by vascular permeability factor/vascular endothelial growth factor. Nat Med. 2001 May; 7 (5): 569-74. doi: 10.1038/87895. PMID: 11329058.
5. Beaulieu J M, Del'guidice T, Sotnikova T D, Lemasson M, Gainetdinov R R. Beyond CAMP: The Regulation of Akt and GSK3 by Dopamine Receptors. Front Mol Neurosci. 2011 Nov. 1; 4:38. doi: 10.3389/fnmol.2011.00038. PMID: 22065948; PMCID: PMC3206544.
6. Beaulieu J M, Tirotta E, Sotnikova T D, Masri B, Salahpour A, Gainetdinov R R, Borrelli E, Caron MG. Regulation of Akt signaling by D2 and D3 dopamine receptors in vivo. J Neurosci. 2007 Jan. 24; 27 (4): 881-5. doi: 10.1523/JNEUROSCI.5074-06.2007. PMID: 17251429; PMCID: PMC6672900.
7. Bourlev V, Volkov N, Pavlovitch S, Lets N, Larsson A, Olovsson M. The relationship between microvessel density, proliferative activity and expression of vascular endothelial growth factor-A and its receptors in eutopic endometrium and endometriotic lesions. Reproduction. 2006 September; 132 (3): 501-9. doi: 10.1530/rep.1.01110. PMID: 16940291.
8. Burney R O, Giudice L C. Pathogenesis and pathophysiology of endometriosis. Fertil Steril. 2012 September; 98 (3): 511-9. doi: 10.1016/j.fertnstert.2012.06.029. Epub 2012 Jul. 20. PMID: 22819144; PMCID: PMC3836682.
9. Canosa S, Moggio A, Brossa A, Pittatore G, Marchino G L, Leoncini S, Benedetto C, Revelli A, Bussolati B. Angiogenic properties of endometrial mesenchymal stromal cells in endothelial co-culture: an in vitro model of endometriosis. Mol Hum Reprod. 2017 Mar. 1; 23 (3): 187-198. doi: 10.1093/molehr/gax006. PMID: 28158750.
10. Cervelló I, Mas A, Gil-Sanchis C, Peris L, Faus A, Saunders P T, Critchley H O, Simón C. Reconstruction of endometrium from human endometrial side population cell lines. PLOS One. 2011; 6 (6): e21221. doi: 10.1371/journal.pone.0021221. Epub 2011 Jun. 21. PMID: 21712999; PMCID: PMC3119688.
12. Dimitrov R, Timeva T, Kyurkchiev D, Stamenova M, Shterev A, Kostova P, Zlatkov V, Kehayov I, Kyurkchiev S. Characterization of clonogenic stromal cells isolated from human endometrium. Reproduction. 2008 April; 135 (4): 551-8. doi: 10.1530/REP-07-0428. PMID: 18367513.
13. Dominici M, Le Blanc K, Mueller I, Slaper-Cortenbach I, Marini F, Krause D, Deans R, Keating A, Prockop Dj, Horwitz E. Minimal criteria for defining multipotent mesenchymal stromal cells. The International Society for Cellular Therapy position statement. Cytotherapy. 2006; 8 (4): 315-7. doi: 10.1080/14653240600855905. PMID: 16923606.
14. Donnez J, Smoes P, Gillerot S, Casanas-Roux F, Nisolle M. Vascular endothelial growth factor (VEGF) in endometriosis. Hum Reprod. 1998 June; 13 (6): 1686-90. doi: 10.1093/humrep/13.6.1686. PMID: 9688413.
15. Dunselman G A, Vermeulen N, Becker C, Calhaz-Jorge C, D'Hooghe T, De Bie B, Heikinheimo O, Horne A W, Kiesel L, Nap A, Prentice A, Saridogan E, Soriano D, Nelen W; European Society of Human Reproduction and Embryology. ESHRE guideline: management of women with endometriosis. Hum Reprod 2014 March; 29 (3): 400-12.
16. Galle PC. Clinical presentation and diagnosis of endometriosis. Obstet Gynecol Clin North Am. 1989; 16 (1): 29-42.
17. Gargett C E, Gurung S. Endometrial Mesenchymal Stem/Stromal Cells, Their Fibroblast Progeny in Endometriosis, and More. Biol Reprod. 2016 June; 94 (6): 129. doi: 10.1095/biolreprod. 116.141325. Epub 2016 May 4. PMID: 27146030.
18. Gargett C E, Schwab K E, Brosens J J, Puttemans P, Benagiano G, Brosens I. Potential role of endometrial stem/progenitor cells in the pathogenesis of early-onset endometriosis. Mol Hum Reprod. 2014 July; 20 (7): 591-8. doi: 10.1093/molehr/gau025. Epub 2014 Mar. 27. PMID: 24674992.
19. Gargett C E, Schwab K E, Deane J A. Endometrial stem/progenitor cells: the first 10 years. Hum Reprod Update. 2016 March-April; 22 (2): 137-63. doi: 10.1093/humupd/dmv051. Epub 2015 Nov. 9. PMID: 26552890; PMCID: PMC4755439.
20. Gargett C E, Schwab K E, Zillwood R M, Nguyen H P, Wu D. Isolation and culture of epithelial progenitors and mesenchymal stem cells from human endometrium. Biol Reprod. 2009 June; 80 (6): 1136-45. doi: 10.1095/biolreprod.108.075226. Epub 2009 Feb. 18. PMID: 19228591; PMCID: PMC2849811.
21. Gibson D A, Simitsidellis I, Collins F, Saunders PT K. Androgens, oestrogens and endometrium: a fine balance between perfection and pathology. J Endocrinol. 2020; 246 (3): R75-R93. doi: 10.1530/JOE-20-0106.
22. Giudice L C, Kao L C. Endometriosis. Lancet. 2004; 364 (9447): 1789-1799. doi: 10.1016/S0140-6736 (04) 17403-5.
23. Gómez R, Abad A, Delgado F, Tamarit S, Simón C, Pellicer A. Effects of hyperprolactinemia treatment with the dopamine agonist quinagolide on endometriotic lesions in patients with endometriosis-associated hyperprolactinemia. Fertil Steril. 2011 Mar. 1; 95 (3): 882-8.e1. doi: 10.1016/j.fertnstert.2010.10.024. Epub 2010 Nov. 5. PMID: 21055747.
24. Han F, Konkalmatt P, Mokashi C, Kumar M, Zhang Y, Ko A, Farino Z J, Asico L D, Xu G, Gildea J, Zheng X, Felder R A, Lee REC, Jose P A, Freyberg Z, Armando I. Dopamine D₂ receptor modulates Wnt expression and control of cell proliferation. Sci Rep. 2019 Nov. 14; 9 (1): 16861. doi: 10.1038/s41598-019-52528-4. PMID: 31727925; PMCID: PMC6856370.
25. Igbokwe R, Jolas T, Waters A, Webster J. Differential effects of dopamine agonists at D₂S and 5HT2B receptors. Endocrine Abstracts. 2009, 19 P251
26. Kao A P, Wang K H, Chang C C, Lee J N, Long C Y, Chen H S, Tsai C F, Hsieh T H, Tsai E M. Comparative study of human eutopic and ectopic endometrial mesenchymal stem cells and the development of an in vivo endometriotic invasion model. Fertil Steril. 2011 Mar. 15; 95 (4): 1308-15.e1. doi: 10.1016/j.fertnstert.2010.09.064. Epub 2010 Nov. 3. PMID: 21047634.
27. Liu Y, Liang S, Yang F, Sun Y, Niu L, Ren Y, Wang H, He Y, Du J, Yang J, Lin J. Biological characteristics of endometriotic mesenchymal stem cells isolated from ectopic lesions of patients with endometriosis. Stem Cell Res Ther. 2020 Aug. 8; 11 (1): 346. doi: 10.1186/s13287-020-01856-8. PMID: 32771033; PMCID: PMC7414689.
28. Masuda H, Anwar S S, Buhring H J, Rao J R, Gargett C E. A novel marker of human endometrial mesenchymal stem-like cells. Cell Transplant. 2012; 21 (10): 2201-14. doi: 10.3727/096368911X637362. Epub 2012 Mar. 27. PMID: 22469435.
29. Masuda H, Matsuzaki Y, Hiratsu E, Ono M, Nagashima T, Kajitani T, Arase T, Oda H, Uchida H, Asada H, Ito M, Yoshimura Y, Maruyama T, Okano H. Stem cell-like properties of the endometrial side population: implication in endometrial regeneration. PLOS One. 2010 Apr. 28; 5 (4): e10387. doi: 10.1371/journal.pone.0010387. PMID: 20442847; PMCID: PMC2860997.
30. McLaren J. Vascular endothelial growth factor and endometriotic angiogenesis. Hum Reprod Update. 2000 January-February; 6 (1): 45-55. doi: 10.1093/humupd/6.1.45. PMID: 10711829.
31. Moggio A, Pittatore G, Cassoni P, Marchino G L, Revelli A, Bussolati B. Sorafenib inhibits growth, migration, and angiogenic potential of ectopic endometrial mesenchymal stem cells derived from patients with endometriosis. Fertil Steril. 2012 December; 98 (6): 1521-30.e2. doi: 10.1016/j.fertnstert.2012.08.003. Epub 2012 Sep. 12. PMID: 22981172.
33. Patyna S, Arrigoni C, Terron A, Kim T W, Heward J K, Vonderfecht S L, Denlinger R, Turnquist S E, Evering W. Nonclinical safety evaluation of sunitinib: a potent inhibitor of VEGF, PDGF, KIT, FLT3, and RET receptors. Toxicol Pathol. 2008 December; 36 (7): 905-16. doi: 10.1177/0192623308326151. Epub 2008 Nov. 3. PMID: 18981453.
35. Pittatore G, Moggio A, Benedetto C, Bussolati B, Revelli A. Endometrial adult/progenitor stem cells: pathogenetic theory and new antiangiogenic approach for endometriosis therapy. Reprod Sci 2014; 21:296-304.
36. Schade R, Andersohn F, Suissa S, Haverkamp W, Garbe E. Dopamine agonists and the risk of cardiac-valve regurgitation. N Engl J Med. 2007 Jan. 4; 356 (1): 29-38. doi: 10.1056/NEJMoa062222. PMID: 17202453.
37. Schultz P N, Ginsberg L, Mccutcheon I E, Samaan N, Leavens M, Gagel R F. Quinagolide in the management of prolactinoma. Pituitary. 2000 December; 3 (4): 239-49. doi: 10.1023/a: 1012884214668. PMID: 11788012.
38. Schwab K E, Chan R W, Gargett C E. Putative stem cell activity of human endometrial epithelial and stromal cells during the menstrual cycle. Fertil Steril. 2005 October; 84 Suppl 2:1124-30. doi: 10.1016/j.fertnstert.2005.02.056. PMID: 16210003.
39. Schwab K E, Gargett C E. Co-expression of two perivascular cell markers isolates mesenchymal stem-like cells from human endometrium. Hum Reprod. 2007 November; 22 (11): 2903-11. doi: 10.1093/humrep/dem265. Epub 2007 Sep. 14. PMID: 17872908.
40. Sinha S, Vohra P K, Bhattacharya R, Dutta S, Sinha S, Mukhopadhyay D. Dopamine regulates phosphorylation of VEGF receptor 2 by engaging Src-homology-2-domain-containing protein tyrosine phosphatase 2. J Cell Sci. 2009 Sep. 15; 122 (Pt 18): 3385-92. doi: 10.1242/jcs.053124. Epub 2009 Aug. 25. PMID: 19706677; PMCID: PMC2736867.
41. Shome S, Dasgupta P S, Basu S. Dopamine regulates mobilization of mesenchymal stem cells during wound angiogenesis. PLOS One. 2012; 7 (2): e31682. doi: 10.1371/journal.pone.0031682. PMID: 22355389; PMCID: PMC3280323.
42. Wang X, Wang Z B, Luo C, Mao X Y, Li X, Yin J Y, Zhang W, Zhou H H, Liu Z Q. The Prospective Value of Dopamine Receptors on Bio-Behavior of Tumor. J Cancer. 2019 Mar. 3; 10 (7): 1622-1632. doi: 10.7150/jca.27780. PMID: 31205518; PMCID: PMC6548012.

1. A method for detecting an ectopic endometrial mesenchymal stromal cell (E-MSC) or for detecting one or more ectopic E-MSCs in a test sample, the method comprising:
probing a test cell or a test sample for the presence or expression of D₂ to determine a level of D₂.

2. The method of claim 1, wherein the test cell or test sample is further probed for:
the presence or expression of endoglin (CD105) to determine a level of endoglin expression; and/or
the presence or expression of platelet derived growth factor receptor beta (PDGFRβ or CD140b) to determine a level of PDGFRβ expression.

3. The method of claim 1 or 2, further comprising:
(i) comparing the level of D₂ detected for the test cell or the test sample with a reference or control amount or level of D₂, wherein the reference or control amount or level of D₂ is derived from a level of D₂ expression detected in a eutopic E-MSC or from a sample comprising eutopic E-MSCs; and/or
(ii) comparing the level of endoglin detected for the test cell or the test sample with a reference or control amount or level of endoglin, wherein the reference or control amount or level of endoglin is derived from a level of endoglin expression detected in a eutopic E-MSC or from a sample comprising eutopic E-MSCs; and/or
(iii) comparing the level of PDGFRβ detected for the test cell or the test sample with a reference or control amount or level of PDGFRβ, wherein the reference or control amount of PDGFRβ is derived from a level of PDGFRβ expression detected in a eutopic E-MSC or a sample comprising eutopic E-MSCs.

4. The method of claim 3, wherein when the level of D₂ and/or endoglin and/or PDGFRβ detected for the test cell or the test sample is:
(a) higher than the reference or control amount or level of D₂, the test cell is determined to be an ectopic E-MSC or the test sample is determined to comprise one or more ectopic E-MSCs; and/or
(b) higher than the reference or control amount or level of endoglin, the test cell is determined to be an ectopic E-MSC or the test sample is determined to comprise one or more ectopic E-MSCs; and/or
(c) lower than the reference or control amount or level of PDGFRβ, the test cell is determined to be an ectopic E-MSC or the test sample is determined to comprise one or more ectopic E-MSCs.

5. The method of any one of claims 1 to 4, further comprising one or more steps in which the test cell or the test sample is further or additionally probed for the presence or expression of one or more mesenchymal, haematopoietic, endometriotic, epithelial and/or endothelial markers.

6. The method of any one of claims 1 to 5, further comprising:
probing the test cell or the test sample for the presence or expression of one or more of the following:
(i) CD44 (a cell-surface glycoprotein involved in cell-cell interactions, cell adhesion and migration)
(ii) Ecto-5′-nucleotidase (CD73);
(iii) Sushi domain-containing protein 2 (SUSD2);
(iv) Integrin, beta 1 subunit (CD29); and
(v) CD90 (Thy-1);
wherein a test cell or test sample shown to express or contain an amount of one or more of the markers (i) to (v) is determined to be or to comprise an ectopic E-MSC.

7. The method of any one of claims 1 to 6, further comprising:
probing the test cell or test sample for the presence or expression of one or more of the following:
(a) melanoma cell adhesion molecule (CD146);
(b) CD34;
(c) RTP Type C (receptor tyrosine phosphatase, CD45);
(d) Epithelial cell adhesion molecule EpCAM and
(e) TEK receptor tyrosine kinase (TIE2);
wherein a test cell or test sample shown to express or contain a low amount or substantially no amount of any one or more of the markers (a)-(e) is determined to be or to comprise an ectopic E-MSC.

8. A method of diagnosing ectopic endometriosis, the method comprising the steps of:
detecting one or more ectopic endometrial mesenchymal stromal cells (E-MSC) in a test sample according to the method of any one of claims 1 to 7;
wherein when the test sample is determined to comprise one or more ectopic E-MSCs, the sample is identified as having been obtained from or provided by a subject who:
(i) is suffering from ectopic endometriosis;
(ii) is susceptible/predisposed to endometriosis; and/or
(iii) is convalescing from or is between occurrences of ectopic endometriosis.

9. A method of diagnosing ectopic endometriosis according to claim 8, the method further comprising a step of allocating a treatment to a subject diagnosed as having or being susceptible to ectopic endometriosis, optionally wherein the method further comprises administering quinagolide to the subject.

10. A kit for detecting ectopic endometrial mesenchymal stromal cells (E-MSCs), said kit comprising an anti-dopamine receptor 2 antibody.

11. The kit of claim 10, wherein the kit further comprises an anti-endoglin antibody (anti-CD105 antibody) and/or anti-PDGFRβ antibody (anti-CD140 antibody).

12. A kit according to any one of claim 10 or 11, further comprising one or more antibodies selected from the following:
(i) an anti-CD44;
(ii) an anti-CD73;
(iii) an anti-CD146;
(iv) an anti-SUSD2;
(v) an anti-CD29;
(vi) an anti-CD90;
(vii) an anti-CD34;
(viii) an anti CD45;
(ix) an anti-EpPCAM; and
(x) an anti-TIEie2; and/or
further comprising one or more of:
(a) buffers;
(b) solutions;
(c) tools;
(d) receptacles; and
(e) instructions for use.

13. A method of staging, assessing or monitoring ectopic endometriosis in a subject, or a method of determining whether or not ectopic endometriosis will re-occur in a subject, said method comprising providing a test sample and determining an amount of D₂ in said sample, wherein the amount of D₂ detected is correlated with or representative of, the stage or progression of ectopic endometriosis in the subject and/or the likelihood of ectopic endometriosis re-occurrence in the subject.

14. The method of claim 13, wherein the sample is provided by:
a healthy subject; or
a subject with ectopic endometriosis; or
a subject being treated for ectopic endometriosis; or
a subject convalescing or recovered from an episode of ectopic endometriosis; or
a subject in remission from ectopic endometriosis.

15. The method of claim 13 or 14, wherein a relatively low amount of D₂ indicates that the sample has been provided by a healthy subject, a subject not suffering from ectopic endometriosis, a subject in remission from ectopic endometriosis and/or a subject responding to treatment for ectopic endometriosis.

16. The method of any one of claims 13 to 15, wherein a relatively high amount of D₂ indicates that the sample has been provided by a subject with ectopic endometriosis, a worsening case of endometriosis and/or ectopic endometriosis which is not responding to treatment.

17. The method of any one of claims 13 to 16, wherein repeat samples are provided by a subject to be tested for an amount of D₂.

18. The method of claim 15 or 16, wherein relatively high or relatively low amounts of D₂ are determined by comparing the amount of D₂ detected in the sample, with a control or reference amount of D₂.

19. The method of claim 18, wherein the reference or known amount of D₂ is indicative of a particular state or stage of ectopic endometriosis.

20. A method of determining whether or not a subject needs prophylactic treatment and/or a hormone-based treatment for ectopic endometriosis, said method comprising providing a test sample from a subject to be considered for prophylactic treatment and/or a hormone-based treatment for ectopic endometriosis and determining an amount of D₂ in said sample, wherein the amount of D₂ detected is correlated with or representative of, the stage or progression of ectopic endometriosis in the subject and/or the likelihood of ectopic endometriosis re-occurrence in the subject.

21. A D₂ agonist for use in treating or preventing ectopic endometriosis.

22. A D₂ agonist for use in containing, controlling, restricting or inhibiting the growth, development or spread of ectopic endometriosis.

23. Use of a D₂ agonist or a D₂ agonist for use, in inhibiting or preventing the invasive properties of ectopic E-MSCs.

24. Use of a D₂ agonist or a D₂ agonist for use, in inhibiting, limiting or preventing the endothelial differentiation of an ectopic E-MSC.

25. A method of inhibiting or preventing the invasive properties of ectopic E-MSCs, said method comprising contacting an ectopic E-MSC with a D₂ agonist.

26. The method of claim 25, wherein the ectopic E-MSC is contacted with an invasion inhibiting or preventing amount of a D₂ agonist.

27. A method of inhibiting, limiting or preventing the endothelial differentiation of an ectopic E-MSC, said method comprising contacting an ectopic E-MSC with a D₂ agonist.

28. The method of claim 27, wherein the ectopic E-MSC is contacted with an endothelial differentiation inhibiting or limiting amount of a D₂ agonist.

29. The method of any one of claim 27 or 28, wherein the method is conducted in an endothelial co-culture model of angiogenesis and said method comprises contacting an ectopic E-MSC with a D₂ agonist in an endothelial co-culture model of angiogenesis.

30. The D₂ receptor agonist for use of any one of claims 21 to 22, the use of claim 23 or 24, or method of any one of claims 25 to 28, wherein the D₂ agonist is to be administered to a subject:
(i) suffering from ectopic endometriosis; or
(ii) susceptible or predisposed to ectopic endometriosis; or
(iii) convalescing from or between occurrences of ectopic endometriosis.

31. The D₂ receptor agonist for use of any one of claims 21 to 22 and 30, use of any one of claims 23 to 24 and 30 or method of any one of claims 25 to 30, wherein the D₂ receptor agonist is quinagolide.

32. Quinagolide for use in treating or preventing ectopic endometriosis.

33. Quinagolide for use in containing, controlling, restricting or inhibiting the growth, development or spread of ectopic endometriosis.

34. Use of quinagolide or quinagolide for use, in inhibiting or preventing the invasive properties of ectopic E-MSCs.

35. Use of quinagolide or quinagolide for use, in inhibiting, limiting or preventing the endothelial differentiation of an ectopic E-MSCs.

36. A method of inhibiting or preventing the invasive properties of ectopic E-MSC, said method comprising contacting an ectopic E-MSC with quinagolide.

37. The method of claim 36, wherein the ectopic E-MSC is contacted with an invasion inhibiting or preventing amount of quinagolide.

38. A method of inhibiting, limiting or preventing the endothelial differentiation of an ectopic E-MSCs, said method comprising contacting an ectopic E-MSC with quinagolide.

39. The method of claim 38, wherein the ectopic E-MSC is contacted with an endothelial differentiation inhibiting or limiting amount of quinagolide

40. The method of any one of claim 38 or 39, wherein the method is conducted in an endothelial co-culture model of angiogenesis and said method comprises contacting an ectopic E-MSC with quinagolide in an endothelial co-culture model of angiogenesis.

41. Quinagolide for use of any one of claims 32 to 35 or the method of any one of claims 36 to 39, wherein the quinagolide is to be administered to a subject:
(i) suffering from ectopic endometriosis;
(ii) susceptible or predisposed to ectopic endometriosis; and/or
(iii) convalescing from or between occurrences of ectopic endometriosis.

42. The use or D₂ agonist for use or method of any one of claim 21 to 28 or 30 or the use or quinagolide for use or method of any one of claim 32 to 39 or 41 wherein the D₂ agonist or quinagolide is formulated for intravaginal administration.

43. The use or D₂ agonist for use or method of any one of claim 21 to 28, 30 or 42 or the use, quinagolide for use or method of any one of claims 32 to 39 or 41 to 42, wherein the D₂ agonist or quinagolide is formulated together with one or more pharmaceutically acceptable excipients, diluents and/or carriers.

44. The use or D₂ agonist for use or method of any one of claim 21 to 28, 30, 42 or 43 or the use, quinagolide for use or method of any one of claims 32 to 39 or 41 to 43, wherein the D₂ agonist quinagolide is comprised within and/or loaded into a polymeric drug-device unit, optionally wherein the polymeric drug-device unit comprises a polyurethane block copolymer obtainable by reacting together:
(a) a poly(alkylene oxide);
(b) a difunctional compound;
(c) a difunctional isocyanate; and
(d) optionally a block copolymer comprising poly(alkylene oxide) blocks.

45. The use, quinagolide for use or method of any one of claims 32 to 39 or 41 to 44, wherein the quinagolide is selected from the group consisting of quinagolide, a pharmaceutically acceptable quinagolide salt, quinagolide hydrochloride, any active enantiomer, the quinagolide hydrochloride enantiomer with absolute configuration 3S,4aS,10aR, the quinagolide metabolite N-desethyl, the quinagolide metabolite N,N-didesethyl and pharmaceutically acceptable salts thereof.

46. The method of any one of claim 26 or 37, wherein the invasion inhibiting amount is between about 1 and about 350 μg/per day of D₂ agonist or quinagolide.

47. The method of any one of claim 28 or 39, wherein the endothelial differentiation inhibiting or limiting amount is between about 1 and about 350 μg/per day of D₂ agonist or quinagolide.
